(19) United States
(12) Patent Application Publication
(10) Pub. No.: US 2009/0220953 A1
(43) Pub. Date: Sep. 3, 2009

(54) IDENTIFICATION OF ANCESTRAL HAPLOTYPES AND USES THEREOF

(75) Inventors: Roger Letts Dawkins, Western Australia (AU); Joseph Frederick Williamson, Western Australia (AU); Craig Anthony McLure, Western Australia (AU)

Correspondence Address: DAVIS WRIGHT TREMAINE, LLP/Seattle, 1201 Third Avenue, Suite 2200, SEATTLE, WA, 98101-3045, US

(73) Assignee: C.Y.O'Connor ERADE Village Foundation, Western Australia (AU)

(21) Appl. No.: 12/064,627

(22) PCT Filed: Aug. 24, 2006
(86) PCT No.: PCT/AU2006/001232

§ 371 Date: Aug. 18, 2008

(30) Foreign Application Priority Data
Aug. 24, 2005 (AU) 2005904603

(51) Int. Cl. C12Q 1/68

(52) U.S. Cl. 435/6; 536/24.33

(57) ABSTRACT

The present invention relates to the identification of haplospecific geometric elements (HGEs) in a multigene cluster comprising genes encoding complement control proteins. The present invention also relates to methods of performing genomic matching techniques (GMT) which enables the identification of HGEs of a duplicated region within a haplotype block. HGEs identified using the methods of the invention can also be analysed to determine if they are markers for a trait of interest such as a disease trait. Furthermore, the present invention relates to methods of determining an individual's susceptibility or predisposition to age-related macular degeneration, recurrent spontaneous abortion, Sjögren's Syndrome and/or psoriasis vulgaris by analysing the genotype of the individual within a multigene cluster comprising genes encoding complement control proteins.

### FIELD OF THE INVENTION

The present invention relates to the identification of haplospecific geometric elements (HGEs) in a multigene cluster comprising genes encoding complement control proteins. The present invention also relates to methods of performing genomic matching techniques (GMT) which enables the identification of HGEs of a duplicated region within a haplotype block. HGEs identified using the methods of the invention can also be analysed to determine if they are markers for a trait of interest such as a disease trait. Furthermore, the present invention relates to methods of determining an individual's susceptibility or predisposition to age-related macular degeneration, recurrent spontaneous abortion, Sjögren's Syndrome and/or psoriasis vulgaris by analysing the genotype of the individual within a multigene cluster comprising genes encoding complement control proteins.

### BACKGROUND OF THE INVENTION

It has been determined that the genome is actually quite uneven in the distribution of critical polymorphic regions. Polymorphic frozen blocks are rich in nucleotide diversity, indels, duplications and disease genes and can be located using appropriate bioinformatic tools (Dawkins et al. 1999).

Ancestral haplotypes are DNA sequences from multigene complexes such as MHC (U.S. Pat. No. 6,383,747). The ancestral haplotypes of the MHC extend from HLA A to HLA DR and beyond (Cattley et al. 2000) have been conserved en bloc. These ancestral haplotypes and recombinants between any two of them account for about 73% of haplotypes in a Caucasian population. The existence of ancestral haplotypes implies conservation of large chromosomal segments. These ancestral haplotypes carry many MHC genes, other than the HLA, which may be relevant to antigen presentation, autoimmune responses and transplantation rejection. Tissue typing is an analysis of the combination of alleles encoded within the MHC. Many of these allelic combinations can be recognised as ancestral haplotypes.

There is a need for identification of further haplospecific geometric elements (HGEs) which can be used in the analysis of ancestral haplotypes. In particular, it is desirable to identify haplospecific geometric elements (HGEs) which can be used as markers for traits of interest. In addition, there is a need for further markers for disease states.

### SUMMARY OF THE INVENTION

The present inventors have identified haplospecific geometric elements (HGEs) within multigene clusters comprising genes encoding complement control proteins that can be used in the analysis of ancestral haplotypes. These HGEs can be used as markers of a trait of interest, and/or used to identify associations between a trait of interest and a genetic locus which in turn can be used to characterize a genetic factor which plays a role in the trait.

In a first aspect, the present invention provides a method of identifying a haplospecific geometric element (HGE) of a region of the genome of an organism comprising a duplication, where the HGE is characteristic of a haplotype block, the method comprising,

i) detecting a region of the genome of an organism which comprises duplicated portions,

ii) comparing the duplicated portions of the region to identify at least one polymorphism between the duplicated portions,

iii) comparing two or more ancestral haplotypes to determine if the polymorphism is the same or different between the duplicated regions of the two or more ancestral haplotypes, and

iv) confirming that the polymorphism is stably transmitted,

wherein a HGE of the region which is characteristic of a haplotype block is polymorphic between the duplicated portions of the region of the haplotype block as well as polymorphic between two or more different ancestral haplotypes, and wherein the HGE forms at least part of a multigene cluster comprising genes encoding complement control proteins.

In a particularly preferred embodiment, the polymorphism between the duplicated portions is a length polymorphism.

Preferably, the length polymorphism is a result of a varying number of insertions and deletions, including repeat units.

The repeat units can be of any length, with individual units not necessarily being exact repeats. In a preferred embodiment the repeat units are di-nucleotide or tri-nucleotide repeats, more preferably complex di-nucleotide or tri-nucleotide repeats which are not all exact repeats.

In another aspect, the present invention provides a method for determining whether the genome of an individual has the same ancestral haplotype as the genome of another individual, the method comprising comparing haplospecific geometric elements (HGEs) within a multigene cluster of each individual, wherein said multigene cluster comprises genes encoding complement control proteins, and said HGEs comprise haplospecific sequences which are specific for a particular ancestral haplotype, and wherein the sequences flanking said HGEs are substantially conserved between ancestral haplotypes.

Preferably, the HGEs were identified using a method of the first aspect of the invention. Thus, it is preferable that the method comprises performing the genomic matching technique.

The comparison can be based on any feature that can be used to distinguish two different nucleic acid sequences. Preferably, said comparison is based on at least one of:

(a) differences in the sequence of said HGEs,

(b) differences in the length of said HGEs,

(c) differences in the number of HGEs, or

(d) differences in the pattern of amplification products of said HGEs.

The comparison could also be based on differences in the primer binding sequence resulting in variations of amplification efficiency between different haplotypes.

In a particularly preferred embodiment, said comparison is at least based on differences in the pattern of amplification products of said HGEs.

Any technique known in the art to characterize nucleic acid sequence or length can be used in the methods of the invention, examples include, but are not limited to, nucleic acid sequence analysis, restriction fragment length polymorphism analysis, reaction with a haplospecific probe, heteroduplex analysis and primer directed amplification. The genome itself may be subject to the analysis or via cDNA or mRNA.

In another embodiment, the method comprises

i) amplifying a region of the multigene cluster comprising genes encoding complement control proteins using at least one set of oligonucleotide primers comprising the following sequences

(SEQ ID NO: 1)

| | |
|---|---|
| a) | 5′ AAT TCC AAA TTG GCC TGG TTG A 3′ |

(SEQ ID NO: 2)

| | |
|---|---|
| and | 5′ CCT TCC CTT TGA GAT GTG GAA CA 3′, |

(SEQ ID NO: 3)

| | |
|---|---|
| b) | 5′ GTC AGC TTG GAT TGC CCT TGG TTC TA 3′ |

(SEQ ID NO: 4)

| | |
|---|---|
| and | 5′ CCT GGG CAA CAA AGC AAG ACA TTG T 3′, |

(SEQ ID NO: 5)

| | |
|---|---|
| c) | 5′ GCC TCT TGG TTT GAT TTT GG 3′ |

(SEQ ID NO: 6)

| | |
|---|---|
| and | 5′ CAG GGT CTA GCA TGA AGA GTA AAA 3′, |

(SEQ ID NO: 7)

| | |
|---|---|
| d) | 5′ GCA AAC TCA ACA TTT CCC TAA CA 3′ |

(SEQ ID NO: 8)

| | |
|---|---|
| and | 5′ TGA TAC CAG GAG AAA TTG CAT 3′, |
| and | |

ii) analysing the amplification products to determine the ancestral haplotype of the individual.

As the skilled person will appreciate, variants of the above-mentioned oligonucleotide primers can also be used to achieve the same result.

With regard to the above embodiment, it is preferred that step ii) comprises analysing the size of the amplification products.

In a preferred embodiment, the genes encoding complement control proteins are located at 1q32 of the human genome. This region is also known in the art as the Regulator of Complement Control (RCA) gene cluster.

In a preferred embodiment, the cluster comprises at least one gene (or pseudogene) selected from, but not limited to, the group consisting of: CR1 (also known as C3b/C4b receptor and CD35), CR1-like protein, membrane cofactor protein (MCP) (also known as CD46), MCP-like protein, CR2 (also known as C3dg receptor and CD21), decay accelerating factor (DAF) (also known as CD56), C4b-binding protein, Complement Factor H (CFH), Complement Factor H Related 1 (CFHL 1), Complement Factor H Related 2 (CFHL2); Complement Factor H Related 3 (CFHL3) and Complement Factor H Related 4 (CFHL4). Preferably, the genes encoding complement control proteins include genes encoding CR1, CR1-like protein, MCP, MCP-like protein, CFH and/or CFHL4.

In a further aspect, the present invention provides a method of detecting a trait in an individual, the method comprising screening an individual for a haplospecific geometric element (HGE) within a multigene cluster linked to the trait, wherein said multigene cluster comprises genes encoding complement control proteins, and said HGE comprise haplospecific sequences which are specific for a particular ancestral haplotype, and wherein the sequences flanking said HGE are substantially conserved between ancestral haplotypes.

Preferably, the HGEs were identified using a method of the first aspect of the invention. Thus, it is preferable that the method comprises performing the genomic matching technique.

The trait can be any trait of interest. In one embodiment, the trait is parentage. In another embodiment, the trait is a disease state, or predisposition thereto.

In one embodiment, the disease state is an inflammatory disease. Examples include, but are not limited to, recurrent spontaneous abortion, psoriasis vulgaris, systemic lupus erythematosus, age related macular degeneration, uveitis, atypical hemolytic uremia syndrome (HUS), Type 1 diabetes, hypothyroidism, celiac disease, myasthenia gravis, multiple sclerosis or Sjögren's syndrome.

In another embodiment, the disease state is susceptibility to an infection. The infection may be by any organism. Preferably, the infection is a bacterial, fungal or viral infection. An example of a viral infection is measles.

In a further embodiment, the disease state is an non-inflammatory disease. Examples include, but are not limited to, haemochromatosis, stroke, embolism, male infertility, renal disease such as chronic hypocomplementemic nephropathy, transplantation disorders, neurodegenerative disorders or thrombotic thrombocytopenic purpura.

In a preferred embodiment, the method comprises

i) amplifying a region of the multigene cluster comprising genes encoding complement control proteins using at least one set of oligonucleotide primers comprising the following sequences

(SEQ ID NO: 1)

| | |
|---|---|
| a) | 5′ AAT TCC AAA TTG GCC TGG TTG A 3′ |

(SEQ ID NO: 2)

| | |
|---|---|
| and | 5′ CCT TCC CTT TGA GAT GTG GAA CA 3′, |

(SEQ ID NO: 3)

| | |
|---|---|
| b) | 5′ GTC AGC TTG GAT TGC CCT TGG TTC TA 3′ |

(SEQ ID NO: 4)

| | |
|---|---|
| and | 5′ CCT GGG CAA CAA AGC AAG ACA TTG T 3′, |

(SEQ ID NO: 5)

| | |
|---|---|
| c) | 5′ GCC TCT TGG TTT GAT TTT GG 3′ |

(SEQ ID NO: 6)

| | |
|---|---|
| and | 5′ CAG GGT CTA GCA TGA AGA GTA AAA 3′, |

(SEQ ID NO: 7)

| | |
|---|---|
| d) | 5′ GCA AAC TCA ACA TTT CCC TAA CA 3′ |

(SEQ ID NO: 8)

| | |
|---|---|
| and | 5′ TGA TAC CAG GAG AAA TTG CAT 3′, |
| and | |

ii) analysing the amplification products to determine the ancestral haplotype of the individual.

As the skilled person will appreciate, variants of the above-mentioned oligonucleotide primers can also be used to achieve the same result.

With regard to the above embodiment, it is preferred that step ii) comprises analysing the size of the amplification products.

Using the method of the first aspect, the inventors have found an association between particular HGEs and an individuals susceptibility or predisposition to psoriasis vulgaris. This observation enables the skilled person to use standard techniques to identify a genetic factor(s) which increases an individuals risk to psoriasis vulgaris.

Thus, in a further aspect the present invention provides a method of identifying a polymorphism linked and/or responsible for, at least in part, an individuals susceptibility or predisposition to psoriasis vulgaris, the method comprising

i) analysing the genotype at one or more loci of the RCA gene cluster on 1q32 of the human genome of individuals with psoriasis vulgaris,

ii) analysing the genotype at one or more loci of the RCA gene cluster on 1q32 of the human genome of individuals who do not have psoriasis vulgaris, and

iii) identifying a polymorphism linked and/or responsible for, at least in part, an individuals susceptibility to psoriasis vulgaris.

Furthermore, the present invention provides a method of determining whether an individual is susceptible or predisposed to psoriasis vulgaris, the method comprising analysing the genotype of the individual within a multigene cluster comprising genes encoding complement control proteins.

In another aspect, the present invention provides a method of diagnosing whether an individual has psoriasis vulgaris, the method comprising analysing the genotype of the individual within a multigene cluster comprising genes encoding complement control proteins.

Preferably, the multigene cluster is located on 1q32 of the human genome.

In one embodiment, the method comprises screening the individual for a polymorphism identified using a method of the invention.

In another embodiment, the method comprises screening the individual for a haplospecific geometric element linked to psoriasis vulgaris using a method of the invention. For instance, haplotypes H1 and H2 detected by the Genomic matching technique as described in the Examples has been shown to be associated with an increased risk to psoriasis vulgaris.

Using the method of the first aspect, the inventors have found an association between particular HGEs and an individuals susceptibility or predisposition to recurrent spontaneous abortion. This observation enables the skilled person to use standard techniques to identify a genetic factor(s) which increases an individuals risk to recurrent spontaneous abortion.

Thus, in another aspect, the present invention provides a method of identifying a polymorphism linked and/or responsible for, at least in part, an individuals susceptibility or predisposition to recurrent spontaneous abortion, the method comprising

i) analysing the genotype at one or more loci of the RCA gene cluster on 1q32 of the human genome of females with recurrent spontaneous abortion,

ii) analysing the genotype at one or more loci of the RCA gene cluster on 1q32 of the human genome of females who have not experienced recurrent spontaneous abortion, and

iii) identifying a polymorphism linked and/or responsible for, at least in part, an individuals susceptibility to recurrent spontaneous abortion.

In a further aspect, the present invention provides a method of determining whether an individual is susceptible or predisposed to recurrent spontaneous abortion, the method comprising analysing the genotype of the individual within a multigene cluster comprising genes encoding complement control proteins.

In yet another aspect, the present invention provides a method of diagnosing whether an individual has recurrent spontaneous abortion, the method comprising analysing the genotype of the individual within a multigene cluster comprising genes encoding complement control proteins.

Preferably, the multigene cluster is located on 1q32 of the human genome.

In one embodiment, the method comprises screening the individual for a polymorphism identified using a method of the invention.

In another embodiment, the method comprises screening the individual for a haplospecific geometric element linked to recurrent spontaneous abortion using a method of the invention. For instance, haplotypes H2 detected by the Genomic matching technique as described in the Examples has been shown to be associated with a decreased risk to recurrent spontaneous abortion.

Using the method of the first aspect, the inventors have found an association between particular HGEs and an individuals susceptibility or predisposition to Sjögren's Syndrome. This observation enables the skilled person to use standard techniques to identify a genetic factor(s) which increases an individuals risk to Sjögren's Syndrome.

Accordingly, in a further aspect the present invention provides a method of identifying a polymorphism linked and/or responsible for, at least in part, an individuals susceptibility or predisposition to Sjögren's Syndrome, the method comprising

i) analysing the genotype at one or more loci of the RCA gene cluster on 1q32 of the human genome of individuals with Sjögren's Syndrome,

ii) analysing the genotype at one or more loci of the RCA gene cluster on 1q32 of the human genome of individuals who do not have Sjögren's Syndrome, and

iii) identifying a polymorphism linked and/or responsible for, at least in part, an individuals susceptibility to Sjögren's Syndrome.

In yet another aspect, the present invention provides a method of determining whether an individual is susceptible or predisposed to Sjögren's Syndrome, the method comprising analysing the genotype of the individual within a multigene cluster comprising genes encoding complement control proteins.

Furthermore, the present invention provides a method of diagnosing whether an individual has Sjögren's Syndrome, the method comprising analysing the genotype of the individual within a multigene cluster comprising genes encoding complement control proteins.

Preferably, the multigene cluster is located on 1q32 of the human genome.

In one embodiment, the method comprises screening the individual for a polymorphism identified using a method of the invention.

In another embodiment, the method comprises screening the individual for a haplospecific geometric element linked to Sjögren's Syndrome using a method of the invention. For instance, haplotypes AH1 and AH3 detected by the Genomic matching technique as described in the Examples has been shown to be associated with an increased risk to Sjögren's Syndrome.

In a preferred embodiment of the methods relating to determining whether an individual is susceptible or predisposed, or diagnosing, psoriasis vulgaris, recurrent spontaneous abortion or Sjögren's Syndrome, the method comprises

i) amplifying a region of the multigene cluster comprising genes encoding complement control proteins using at least one set of oligonucleotide primers comprising the following sequences

(SEQ ID NO: 1)

| | |
|---|---|
| a) | 5′ AAT TCC AAA TTG GCC TGG TTG A 3′ |

(SEQ ID NO: 2)

| | |
|---|---|
| and | 5′ CCT TCC CTT TGA GAT GTG GAA CA 3′, |

(SEQ ID NO: 3)

| | |
|---|---|
| b) | 5′ GTC AGC TTG GAT TGC CCT TGG TTC TA 3′, |

(SEQ ID NO: 4)

| | |
|---|---|
| | 5′ CCT GGG CAA CAA AGC AAG ACA TTG T 3′, |
| and | |

ii) analysing the amplification products to determine the ancestral haplotype of the individual.

As the skilled person will appreciate, variants of the above-mentioned oligonucleotide primers can also be used to achieve the same result.

With regard to the above embodiment, it is preferred that step ii) comprises analysing the size of the amplification products.

Using the method of the first aspect, the inventors have also found an association between particular HGEs and an individuals susceptibility or predisposition to age-related macular degeneration. Surprisingly, the inventors have found that the genomic matching technique can be more informative than analysing known SNPs associated with age-related macular degeneration. This observation enables the skilled person to use standard techniques to identify a genetic factor(s) which increases an individuals risk to age-related macular degeneration.

Thus, in a further aspect the present invention provides a method of identifying a polymorphism linked and/or responsible for, at least in part, an individuals susceptibility or predisposition to age-related macular degeneration, the method comprising

i) analysing the genotype at one or more loci of the RCA gene cluster on 1q32 of the human genome of individuals with age-related macular degeneration,

ii) analysing the genotype at one or more loci of the RCA gene cluster on 1q3² of the human genome of individuals who do not have age-related macular degeneration, and

iii) identifying a polymorphism linked and/or responsible for, at least in part, an individuals susceptibility to age-related macular degeneration,

wherein the polymorphism is not a polymorphism of the complement factor H gene.

Furthermore, the present invention provides a method of determining whether an individual is susceptible or predisposed to age-related macular degeneration, the method comprising analysing the genotype of the individual within a multigene cluster comprising genes encoding complement control proteins, and wherein the method comprises screening the individual for a haplospecific geometric element linked to age-related macular degeneration.

Also provided is a method of diagnosing whether an individual has age-related macular degeneration, the method comprising analysing the genotype of the individual within a multigene cluster comprising genes encoding complement control proteins, and wherein the method comprises screening the individual for a haplospecific geometric element linked to age-related macular degeneration.

Preferably, the multigene cluster is located on 1q32 of the human genome.

In one embodiment, the method comprises screening the individual for a polymorphism identified using a method of the invention.

Preferably, the haplospecific geometric elements are present in the complement factor H and the complement factor HL4 genes.

In a further preferred embodiment, the method comprises

i) amplifying a region of the complement factor H and the complement factor HL4 genes using at least one set of oligonucleotide primers comprising the following sequences

(SEQ ID NO: 5)

| | |
|---|---|
| a) | 5′ GCC TCT TGG TTT GAT TTT GG 3′ |

(SEQ ID NO: 6)

| | |
|---|---|
| and | 5′ CAG GGT CTA GCA TGA AGA GTA AAA 3′, |

(SEQ ID NO: 7)

| | |
|---|---|
| b) | 5′ GCA AAC TCA ACA TTT CCC TAA CA 3′ |

(SEQ ID NO: 8)

| | |
|---|---|
| and | 5′ TGA TAC CAG GAG AAA TTG CAT 3′, |
| and | |

ii) analysing the amplification products to determine the ancestral haplotype of the individual.

As the skilled person will appreciate, variants of the above-mentioned oligonucleotide primers can also be used to achieve the same result.

With regard to the above embodiment, it is preferred that step ii) comprises analysing the size of the amplification products.

The present inventors have also identified that the method of the first aspect can be used to predict whether an individual is susceptible or predisposed to progress from dry age-related macular degeneration to wet age-related macular degeneration.

Accordingly, in a further aspect the present invention provides a method of determining whether an individual is susceptible or predisposed to progress from dry age-related macular degeneration to wet age-related macular degeneration, the method comprising analysing the genotype of the individual within a multigene cluster comprising genes encoding complement control proteins, and wherein the method comprises screening the individual for a haplospecific geometric element linked to age-related macular degeneration.

Preferably, the haplospecific geometric elements are present in the complement factor H and the complement factor HL4 genes.

In a further preferred embodiment, the method comprises

i) amplifying a region of the complement factor H and the complement factor HL4 genes using at least one set of oligonucleotide primers comprising the following sequences

(SEQ ID NO: 5)

| | |
|---|---|
| a) | 5′ GCC TCT TGG TTT GAT TTT GG 3′ |

(SEQ ID NO: 6)

| | |
|---|---|
| and | 5′ CAG GGT CTA GCA TGA AGA GTA AAA 3′, |

(SEQ ID NO: 7)

| | |
|---|---|
| b) | 5′ GCA AAC TCA ACA TTT CCC TAA CA 3′ |

(SEQ ID NO: 8)

| | |
|---|---|
| and | 5′ TGA TAC CAG GAG AAA TTG CAT 3′, |
| and | |

ii) analysing the amplification products to determine the ancestral haplotype of the individual.

As the skilled person will appreciate, variants of the above-mentioned oligonucleotide primers can also be used to achieve the same result.

With regard to the above embodiment, it is preferred that step ii) comprises analysing the size of the amplification products.

In a further preferred embodiment, the presence of ancestral haplotype 1 (AH1) indicates that the individual has a greater chance of progressing from dry age-related macular degeneration to wet age-related macular degeneration than an individual lacking AH1.

The methods of the invention will typically be performed on a sample obtained from the organism (individual). Preferably, the sample is any biological material which comprises genomic DNA. Examples of such samples include, but are not limited to, blood, serum, plasma, buccal swab, hair follicles, and saliva.

The methods of the invention can be performed on a sample obtained from any organism (individual) which has a genome comprising a multigene cluster comprising genes encoding complement control proteins. Preferably, the organism is a vertebrate, more preferably a mammal. In a particularly preferred embodiment, the mammal is a human. Preferred non-human animals include domestic animals such as sheep, cattle and horses, and companion animals such as cats and dogs.

In a further aspect, the present invention provides an oligonucleotide primer for use in performing a genomic matching technique, wherein the primer can be used to amplify a region of a multigene cluster comprising genes encoding complement control proteins.

Preferably, the primer is selected from:

a) an oligonucleotide comprising a sequence selected from:

(SEQ ID NO: 1)

5′ AAT TCC AAA TTG GCC TGG TTG A 3′,

(SEQ ID NO: 2)

5′ CCT TCC CTT TGA GAT GTG GAA CA 3′,

(SEQ ID NO: 3)

5′ GTC AGC TTG GAT TGC CCT TGG TTC TA 3′,

(SEQ ID NO: 4)

5′ CCT GGG CAA CAA AGC AAG ACA TTG T 3′,

(SEQ ID NO: 5)

5′ GCC TCT TGG TTT GAT TTT GG 3′,

(SEQ ID NO: 6)

5′ CAG GGT CTA GCA TGA AGA GTA AAA 3′,

(SEQ ID NO: 7)

5′ GCA AAC TCA ACA TTT CCC TAA CA 3′
and

(SEQ ID NO: 8)

5′ TGA TAC CAG GAG AAA TTG CAT 3′,

b) an oligonucleotide comprising a sequence which is the reverse complement of any oligonucleotide provided in a), and

c) a variant of a) or b) which can be used to amplify the same region of the human genome as any one of the oligonucleotides of a) or b).

Also provided is a composition comprising an oligonucleotide of the invention and an acceptable carrier.

In a further aspect, the present invention provides a kit comprising an oligonucleotide of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### Key to Sequence Listing

SEQ ID NO's 1 to 10—Oligonucleotide primers.
SEQ ID NO's 11 to 18—Sequences of polynucleotides amplified, or capable of being amplified by the FH1 primer pair (see FIG. 10).

### BRIEF DESCRIPTION OF THE ACCOMPANYING DRAWINGS

FIG. 1. Multiple binding and amplification by primer pairs. Schematic representation of the genomic region on 1q32 showing the duplicated segments containing the CR1 and MCP genes. The lines indicate the positions of the forward (CR1MCP 5) and reverse primers (CR1MCP 6) designated P5+6. The amplified sequences of CR1 and CR1-like have been aligned to show conserved regions flanking a polymorphic geometric element containing multiple complex components which distinguish CR1 and CR1-like sequences. Black shading and white text indicates conserved sequence. Numbers above and below the alignment represent nucleotide positions of CR1-like (Celera-NT_{—}086601) and CR1 (NCBI-NT_{—}021877.16) respectively. Also shown are locations of primers P 11+12 and BstN1 cutting sites (see Table 1). Conserved nucleotides at CR1-like positions 289-391 are part of a L1 element.

FIG. 2. Sequencing reveals the complexity of the haplospecific element and differences between CR1 and CR1-like. Sequence alignment identifies potential indels and polymorphic elements. The TC-rich region is highly polymorphic in keeping with other haplospecific elements. Black shading and white text indicates consensus sequence on either side of the indel polymorphic region. The differences between CR1-like and CR1 are (i) G at 101, 105, 109, 113, 126 and 130 (*); (ii) length differences between 102 and 281 bp; (iii) other indels. For the purposes of classifying the sequences of products we used (i) with or without the remainder. Numbers above and below the alignment represent nucleotide position of CR1-like (Celera-NT_{—}086601) and CR1 (NCBI-NT_{—}021877.16) respectively. Note “Y” indicates nucleotide C/T.

FIG. 3. Segregation of ancestral haplotypes. GMT P5+6 profiles from 3-generation families confirm unequivocal segregation of haplotypes. In each case the profile overlay has been restricted to 2 generations. Individual profiles are coloured as shown in the family tree and the laboratory specimen codes. The number assigned to each band is derived from FIG. 4.

FIG. 4. Genomic polymorphism within the CR1/MCP duplicons. GMT P5+6 profiles following polyacrylamide gel separation were overlayed using internal molecular weight markers of 242, 331 and 404 bp (solid vertical lines) Amplicons differ between individuals (broken vertical lines). Bands have been assigned numbers from the smallest (1) to the largest (19). Some such as 8 are rare in Caucasians

FIG. 5. Reproducibility of the GMT profiles. GMT P5+6 profiles using different PCR conditions demonstrate the reproducibility of the method. The internal markers are as in FIG. 4.

FIG. 6. CR1.02 and CR1.08 haplotype frequencies differ in different clinical groups. RCA-C Recurrent Spontaneous Abortion control group; RCA-P—Recurrent Spontaneous Abortion; HCT—Haemochromatosis; PV—Psoriasis Vulgaris; ARL-C—Adelaide Research Laboratories control group; SLE-P—Systemic Lupus Erythematosus; SS—Sjögren's Syndrome; AH 02=Ancestral Haplotype 02-P5+6=4,0;P11+12=1,13;BstN1-G is rare in RSA but common in PV whereas AH 08—Ancestral Haplotype 08-P5+6=6,13;P11+12=5,11;BstN1-T shows the opposite. Although less dramatic, the binomial probability mass function (EXCEL) shows a decrease in 02 (p=0007) and an increase in 08 (p=002) when RSA-S is compared to RSA-C.

FIG. 7. Polymorphisms within SCR subfamilies. CCPs such as CR1, CR1-like and Crry contain Short Consensus Repeats (Hourcade et al. 1989) which we have classified into subfamilies as a, b, c etc (McLure et al. 2004a; McLure et al. 2004b; McLure et al. 2005a). Each CCP has its particular order such as (ajejbkd)₅ ch in the case of CR1 (McLure et al. 2005a) but the subfamilies are remarkably conserved as indicated by the degree of shading. Some of the known SNPs (Birmingham et al. 2003; Moulds et al. 2001; Xiang et al. 1999) have been mapped to the subfamilies since those changing conserved residues are likely to have profound functional effects. SNPs within a, j or e are likely to alter ligand binding (Birmingham et al. 2003). The BstN1 site is within j. Key: ̂ Translated from the mRNA sequence but absent in respective protein sequence. Hosa is Homo sapien, Mumu is Mus musculus, Rano is Rattus norvegicus, Patr is Pan troglodytes, Paha is Papio hamadryas and Pacy is Papio Cynocephalus.

FIG. 8. Phenotypic proportions of A) CR1-AH1, B) CR1 AH3, C) HLA-DR3 and D) HLA-DR2 haplotypes by Ro/La autoantibody subgroups within pSS. There were 115 pSS patients in the study: 18 were Seronegative, 19 with anti-Ro only, 22 with anti-Ro+La (ppt−) and 56 with anti-Ro+La (ppt(+).

FIG. 9. CR1 AH1 genotype distribution in HLA B8-DR3 positive compared to DR3 negative pSS patients. There is an apparent epistatic interaction between the MHC and CR1 as AH1 positive genotypes are significantly more frequent in individuals who are also positive for HLA B8-DR3 (p=0.033).

FIG. 10. Alignment of sequence from products 50, 55, 60, 11, 18 and 16 generated with FH1 primer pair. CFH copy 1 and copy 2 were obtained from the NCBI Genomic Database NT_{—}004487.17 (http://www.ncbi.nlm.nih.gov/). Sequences provided as SEQ ID NO's 11 to 18 respectively. Forward and reverse primers are underlined.

FIG. 11. Complement related genes on human chromosome 1q21-q32.

FIG. 12. Imperfect duplication and degeneracy of duplicated segment within the RCA b Block. Dot plot comparative analysis of the genomic region containing CFH, CFHL1, CFHL2, CFHL3, CFHL4, CFHL5 and F13B at 1q32 identifies imperfect duplication and gene degeneracy. Duplicated segments share many complex elements, as shown in the magnified region comparing regions of CFH (77.4 kb-79 kb) and CFHL4 (245.6 kb-247.1 kb). These regions share conserved flanking regions but differ markedly within the central or variable region. In this instance there are two variable regions with a central conserved region. Primer pairs FH1 and FH4 have been designed to amplify both of these regions by designing primers in opposite directions within the central region. The proximity of the complex elements to the CFH exon 9 SNP T1277C associated with Age Related Macular Degeneration is also shown.

FIG. 13. Polymorphism and complexity (a) Alignment of the conserved flanking regions of the complex elements from respective sequences of CFH and CFHL4 taken from the NCBI and Celera assemblies. Primer pairs FH1 and FH4 are shown under the green and orange arrows. (b) Sequence of the 6 bands extracted from the agarose gels. Only the polymorphic sequences are shown. This illustrates the complexity of the FH1 element, ie there are a number of repetitive elements (CCTT, TTCT, CT, TTTC, CTAC and CTTC), each varying in copy number. The combination and number of these elements creates the variation seen in the size of the individual amplicons.

FIG. 14. Sequence specific priming within CFH exon 9 and digestion by NLA III. Detection of the CFH T1277C SNP for comparison and association with the haplotypes generated by FH1 and FH4 primers. CFH exon 9 homologues were identified and sequences from the NCBI and Celera assemblies were aligned. The forward and reverse primers were designed to amplify CFH only. Binding of either the forward or reverse primer (sequences above the arrows) within other homologues does occur but CFH exon 9 is the only locus to be efficiently amplified in both the forward and reverse directions.

FIG. 15. Determination of T1277C SNP. Following SSP amplification and digestion with enzyme NLAIII (New England Biolabs) (recognition CATG), C/T homozygotes and heterozygotes are readily distinguished on a polyacrylamide gel. These were confirmed by sequencing exon 9 of the CFH gene from each of these individuals (shown on the right).

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

A “haplotype” is the particular combination of alleles (usually identified by single nucleotide polymorphisms (SNPs)) on one chromosome or a part of a chromosome. Haplotypes can be exploited for the fine mapping of disease genes. A new mutation responsible for a genetic disease always enters the population within an existing haplotype, which is termed the ancestral haplotype. Over several generations, recombination events may occur within the haplotype but the disease allele and the closest SNPs still tend to be inherited as a group. When this haplotype can be identified in a group of patients with the disease, typing the alleles within the haplotype allows a conserved region to be identified, which pinpoints the mutation responsible for the disease. Due to the abundance of SNPs, this technique has the potential to map genes very accurately.

Some SNPs may be in linkage disequilibrium and are inherited in blocks. A “haplotype block” (also known in the art as a “frozen block”) is thus a discrete chromosome region of high linkage disequilibrium (LD) and low haplotype diversity. It is expected that all pairs of polymorphisms within a block will be in strong linkage disequilibrium, whereas other pairs will show much weaker association. Blocks are hypothesized to be regions of low recombination flanked by recombination hotspots. Blocks may contain a large number of SNPs, but a few SNPs are enough to uniquely identify the haplotypes in a block. The HapMap is a map of these haplotype blocks and the specific SNPs that identify the haplotypes are called tag SNPs.

An “ancestral haplotype” block is passed from generation to generation just like familial haplotype blocks but is found at higher than expected frequencies in the population at large between people not closely related, namely all arising from some distant ancestor.

“Haplospecific geometric elements” (HGEs) are geometric in that there is a mathematical relationship between the number of bases which is a characteristic of each ancestral haplotype. There is also geometry in the sense that there is a symmetry around the center of the region which is defined from the boundaries which are more or less common to different ancestral haplotypes. HGEs are also distinctive in that there is non-random usage of nucleotides with iteration of certain components of the sequence. While these components may contain simple sets (eg di and trinucleotide iterations), these do not themselves define the elements and do not allow recognition of haplospecificity or geometric patterns. While HGEs are characteristic of each individual ancestral haplotype, and characterisation thereof therefore provides direct information as to ancestral haplotype, nucleotide sequences outside of the HGEs may also be utilised to distinguish between ancestral haplotypes. Ancestral haplotype sequences differ from one another along their length notwithstanding that marked variation occurs within HGEs. Accordingly, the nucleotide sequence of different ancestral haplotypes may be ascertained and the respective differences therebetween used to construct polynucleotide probes which discriminate between ancestral haplotypes. It is important to appreciate that the sequences flanking HGEs are generally highly conserved between the various ancestral haplotypes. These regions thus allow polynucleotide probes to be produced which allow characterization of HGEs by amplification of such sequences utilizing techniques well known in the art.

The “Genomic matching technique” (GMT) is based on generating haplotype markers with a single primer pair which amplifies duplicated sites. A single test identifies maternal and paternal haplotypes of sequences of up to several hundred kilobases. Within this sequence are multiple linked polymorphisms, both coding and non coding, indels and duplications. Thus, differences in copy number and regulation can be detected and, in this way, there is more information than with the alternative tests.

As used herein, the term “multigene cluster” refers a region of the genome that comprises a high concentration of genes and/or pseudogenes. Typically, many genes of a multigene cluster are interrelated, and have arisen through duplication events. A particularly preferred multigene cluster of the invention is the Regulator of Complement Activation (RCA) gene cluster located in the long arm of chromosome 1 (1q32) of the human genome (de Cordoba et al. 1999).

A “complement control protein” (CCP) is involved in complement regulation, and often have one or more stretches of a common short consensus repeat encoding a 60 amino acid domain. CCPs are found in clusters around the genome including the MHC where they are within the early complement components C2 and Bf, however, the major cluster in the human genome is the Regulator of Complement Activation (RCA) gene cluster. Examples of CCPs include CR1, CR1-like protein, MCP, factor H, C4 binding protein, decay accelerating factor, membrane cofactor protein, and several complement receptors. Further examples are described by de Cordoba et al. (1999).

As used herein, a “duplicated portion” of a region of the genome of an organism refers to a particular sequence being repeated within a haplotype block. The duplication is not an exact copy, however copies of the repeated sequence share significant sequence identity. In one embodiment, the duplicated portions are at least 50%, more preferably at least 60%, more preferably at least 65%, more preferably at least 70%, more preferably at least 75%, more preferably at least 80%, more preferably at least 85%, more preferably at least 90%, more preferably at least 92%, more preferably at least 95%; more preferably at least 97%, and even more preferably at least 99% identical to each other. In another embodiment, one duplicated portion is able to hybridize to the reverse complement of the other duplicated portion under stringent conditions. The duplicated portions may be as few as a hundred base pairs in length or be as large as hundreds of kilobase pairs in length. The duplicated portions may be tandemly duplicated or separated by an unrelated sequence. The duplicated portions may be genes, pseudogenes and/or include inter- or intra-genic, non-coding regions. Duplicated portions of a region can be identified using any technique known in the art. For example, the dot-matrix program described by Sonnhammer and Durbin (1995) can be used to identify duplicated portions of the genome.

The % identity of a polynucleotide is determined by GAP (Needleman and Wunsch, 1970) analysis (GCG program) with a gap creation penalty=8, and a gap extension penalty=3. The query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. Even more preferably, the query sequence is at least 300 nucleotides in length and the GAP analysis aligns the two sequences over a region of at least 300 nucleotides.

As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

The term “polymorphism” refers to the coexistence of more than one form of a locus of interest. A region of the genome of which there are at least two different forms, i.e., two different nucleotide sequences, is referred to as a “polymorphic region” or “polymorphic locus”. A polymorphic locus can be a single nucleotide, the identity of which differs in the other alleles. A polymorphic locus can also be more than one nucleotide long. The allelic form occurring most frequently in a selected population is often referred to as the reference and/or wild-type form. Other allelic forms are typically designated or alternative or variant alleles. Diploid organisms may be homozygous or heterozygous for allelic forms. A diallelic or biallelic polymorphism has two forms. A trialleleic polymorphism has three forms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

As used herein, the phrase “substantially conserved” when referring to sequences flanking a HGE is used as a relative term such that between different individuals of a species the flanking regions are more highly conserved that than the sequences of the HGEs.

The term “linkage” describes the tendency of genes, alleles, loci or genetic markers to be inherited together as a result of their location on the same chromosome. It can be measured by percent recombination between the two genes, alleles, loci, or genetic markers. The term “linkage disequilibrium” refers to a greater than random association between specific alleles at two marker loci within a particular population. In general, linkage disequilibrium decreases with an increase in physical distance. If linkage disequilibrium exists between two markers within one gene, then the genotypic information at one marker can be used to make probabilistic predictions about the genotype of the second marker.

The “sample” refers to a material which comprises the subject's genomic DNA, or RNA encoding a gene of interest. The sample can be used as obtained directly from the source or following at least one step to at least partially purify DNA or RNA from the sample obtained directly from the source. Preferably, the sample comprises genomic DNA. The sample can be prepared in any convenient medium which does not interfere with the methods of the invention. Typically, the sample is an aqueous solution or biological fluid as described in more detail below. The sample can be derived from any source, such as a physiological fluid, including blood, serum, plasma, saliva, sputum, ocular lens fluid, sweat, faeces urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, buccal swab, amniotic fluid, and the like. Herein, fluid homogenates of cellular tissues such as, for example, hair, skin and nail scrapings, meat extracts are also considered biological fluids. Pretreatment may involve preparing plasma from blood, diluting viscous fluids, and the like. Methods of treatment can involve filtration, distillation, separation, concentration, inactivation of interfering components, and the addition of reagents. The selection and pretreatment of biological samples prior to testing is well known in the art and need not be described further.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of about 1 kb on either end such that the gene corresponds to the length of the full-length mRNA. Regions further distances (than about 1 kb) from the coding region may also comprise part of a gene if they directly influence transcription. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. A genomic form or clone of a gene contains the coding region which is interrupted with non-coding sequences termed “introns” or “intervening regions” or “intervening sequences”. Introns are segments of a gene which are transcribed into nuclear RNA (nRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Age-Related Macular Degeneration” (AMD) is an degenerative eye disease that causes damage to the macula (central retina) of the eye. AMD is the leading cause of vision loss in our senior population. Macular Degeneration impairs central vision. The macula is the central part of the retina at the back of the eye that allows us to see fine details clearly. There are two stages of macular degeneration. The Dry Stage is the more common form. In this type of macular degeneration, the delicate tissues of the macula become thinned and slowly lose function. The Wet Stage is less common, but is typically more damaging. The wet type of macular degeneration is caused by the growth of abnormal blood vessels behind the macula. The abnormal blood vessels tend to hemorrhage or leak, resulting in the formation of scar tissue if left untreated. In some instances, the dry stage of macular degeneration can turn into the wet stage.

### Haplospecific Geometric Elements and the Identification Thereof

The inventors have identified polymorphic regions within an ancestral haplotype of a multigene cluster comprising genes encoding complement control proteins which comprises stable stretches of nucleotides which differ between different ancestral haplotypes. These polymorphic regions are haplospecific geometric elements (HGEs).

As will be described herein, HGEs have been shown to occur at various sites within a multigene cluster comprising genes encoding complement control proteins. Elements at each of these sites may be related to each other in that they have the same or predictable geometry.

It should be appreciated that the detection of HGEs, and indeed the characterisation of nucleic acid sequences corresponding to ancestral haplotypes or recombinants thereof are not dependent upon the use of any specific technique. As described herein, a variety of techniques can be used for identification and characterisation of ancestral haplotype specific sequences.

While HGEs are characteristic of each individual ancestral haplotype, and characterisation thereof therefore provides direct information as to ancestral haplotype, nucleotide sequences outside of the HGEs may also be utilised to distinguish between ancestral haplotypes. Ancestral haplotype sequences differ from one another along their length notwithstanding that marked variation occurs within HGEs. Accordingly, the nucleotide sequence of different ancestral haplotypes may be ascertained and the respective differences therebetween used to construct polynucleotide probes which discriminate between ancestral haplotypes. Preferably, the probes hybridize to complementary sequences in a region flanking the HGE and will hybridize to complementary sites represented at least twice.

Single primer sequences may be utilised for amplification (such as linear amplification) whereafter amplified products may be detected by hybridisation with probes complementary in sequence to said amplified HGE.

Paired nucleotide sequences flanking HGEs may be used to amplify the HGEs following multiple cycles of primer extension. Amplified products may be detected by direct visual analysis after fractionation on a gel or other separation medium.

HGEs, or indeed other regions of the ancestral haplotype of the multigene cluster comprising genes encoding complement control proteins may be amplified by direct amplification of single stranded RNA or denatured double stranded DNA

HGEs of characteristic nucleotide sequence are carried by each ancestral haplotype. As a consequence, HGEs are characteristic of each ancestral haplotype of a multigene cluster comprising genes encoding complement control proteins. As previously mentioned, HGEs possess geometry in the sense that there is a symmetry around the centre of the region which is defined from the boundaries which are more or less common to different ancestral haplotypes. HGEs are also distinctive in that there is non-random usage of nucleotides with iteration of certain components of the sequence, for example, but not limited to, complex arrangements of di, tri and tetranucleotide iterations.

HGEs are preferably characterised by possessing conserved sequences at their boundaries and a variant number of di and trinucleotide repeats in the central region.

Preferred primers of the present invention are those set forth below in the 5′ to 3′ direction:

(SEQ ID NO: 1)

| | |
|---|---|
| CR1MCP5: | 5′ AAT TCC AAA TTG GCC TGG TTG A 3′, |

(SEQ ID NO: 2)

| | |
|---|---|
| CR1MCP6: | 5′ CCT TCC CTT TGA GAT GTG GAA CA 3′, |

(SEQ ID NO: 3)

| | |
|---|---|
| CR1MCP11: | 5′ GTC AGC TTG GAT TGC CCT TGG TTC TA 3′, |

(SEQ ID NO: 4)

| | |
|---|---|
| CR1MCP12: | 5′ CCT GGG CAA CAA AGC AAG ACA TTG T 3′, |

(SEQ ID NO: 5)

| | |
|---|---|
| FHF1: | 5′ GCC TCT TGG TTT GAT TTT GG 3′, |

(SEQ ID NO: 6)

| | |
|---|---|
| FHR1: | 5′ CAG GGT CTA GCA TGA AGA GTA AAA 3′, |

(SEQ ID NO: 7)

| | |
|---|---|
| FHF4: | 5′ GCA AAC TCA ACA TTT CCC TAA CA 3′, |
| and | |

(SEQ ID NO: 8)

| | |
|---|---|
| FHR4: | 5′ TGA TAC CAG GAG AAA TTG CAT 3′, |

as well as a variants of any one or more thereof.

In yet another embodiment of the present invention, the identification of an ancestral haplotype can be accomplished by multiple priming using one primer or a set of primers (for example using each of the four above-mentioned primers). According to this embodiment of the invention, there is provided a method for identifying an ancestral haplotype on the genome of an individual comprising amplifying multiple regions within said haplotype with a single primer or set of primers and comparing the amplification products with a reference panel of ancestral haplotypes or with the amplification products from another individual.

The stable transmission of a polymorphism can be detected using any technique known in the art. For example, the polymorphism is analysed in different members of a family to ensure that it is faithfully inherited.

### Oligonucletide Primers

As the skilled address would be aware, the sequence of the oligonucleotide primers described herein can be varied to some degree without effecting their usefulness for the methods of the invention. A variant of an “oligonucleotide” (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they stilt hybridise to the target region. Furthermore, a few nucleotides may be substituted without influencing the ability of the oligonucleotide to hybridise the target region. In addition, variants may readily be designed which hybridise close (for example, but not limited to, within 50 nucleotides) to the region of the genome where the specific oligonucleotides defined herein hybridise. Oligonucleotides can be naturally occurring or synthetic, but are typically prepared by synthetic means.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase) in an appropriate buffer and at a suitable temperature. The length of a primer may vary but typically ranges from 15 to 30 nucleotides. A primer need not match the exact sequence of a template, but must be sufficiently complementary to hybridize with the template.

The term “primer pair” refers to a set of primers including an upstream primer that hybridizes with the 3′ end of the complement of the nucleic acid to be amplified and a downstream primer that hybridizes with the 3′ end of the sequence to be amplified.

The term primer, as defined herein, is meant to encompass any nucleic acid that is capable of priming the synthesis of a nascent nucleic acid in a template-dependent process. Primers may be provided in double-stranded or single-stranded form, although the single-stranded form is preferred. Methods of primer design are well-known in the art, based on the design of complementary sequences obtained from standard Watson-Crick base-pairing (i.e., binding of adenine to thymine or uracil and binding of guanine to cytosine). Computerized programs, when provided with suitable information regarding a target region, for selection and design of amplification primers are available from commercial and/or public sources well known to the skilled artisan.

The primers used in the method of the invention preferably consists of a sequence of at least about 15 consecutive nucleotides, more preferably at least about 18 nucleotides.

Primers used in the methods of the invention can have one or more modified nucleotides. Many modified nucleotides (nucleotide analogs) are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases. Such modifications are well known in the art.

Chimeric primers can also be used. Chimeric primers are primers having at least two types of nucleotides, such as both deoxyribonuucleotides and ribonucleotides, ribonucleotides and modified nucleotides, two or more types of modified nucleotides, deoxyribonucleotides and two or more different types of modified nucleotides, ribonucleotides and two or more different types of modified nucleotides, or deoxyribonucleotides, ribonucleotides and two or more different types of modified nucleotides. One form of chimeric primer is peptide nucleic acid/nucleic acid primers. For example, 5′-PNA-DNA-3′ or 5′-PNA-RNA-3′ primers may be used for more efficient strand invasion and polymerization invasion. Other forms of chimeric primers are, for example, 5′-(2′-O-Methyl) RNA-RNA-3′ or 5′-(2′-O-Methyl) RNA-DNA-3′.

Primers may be chemically synthesized by methods well known within the art. Chemical synthesis methods allow for the placement of detectable labels such as fluorescent labels, radioactive labels, etc. to be placed virtually anywhere within the sequence. Solid phase methods as well as other methods of oligonucleotide or polynucleotide synthesis known to one of ordinary skill may used within the context of the disclosure.

### Genetic Screening

The methods of the invention can be used to identify an association between a locus and a trait of interest. Based on the identified association, the skilled person can use standard techniques to determine whether a particular polymorphism is responsible (at least in part) for the trait, or is linked (in linkage disequilibrium) with a locus that is responsible (at least in part) for the trait.

If the polymorphism is responsible (at least in part) for the trait, the methods of the invention based on the analysis of ancestral haplotypes can be used to detect the trait, or a predisposition thereto, in an individual. Alternatively, once an association is identified other genetic screening techniques can be used that directly target the polymorphism of interest (such as DNA sequencing).

If the polymorphism is linked (in linkage disequilibrium) with a locus that is responsible (at least in part) for the trait, the methods of the invention based on the analysis of ancestral haplotypes can also be used to detect the trait, or a predisposition thereto, in an individual. However, in a preferred embodiment further analysis is performed to map and locate the genetic elements responsible (at least in part) for the trait. Such analysis can be performed using techniques known in the art. In this situation, genetic screening techniques other than those based on the determination of ancestral haplotypes can be used that directly target the polymorphism of interest (such as DNA sequencing).

Genetic assay methods useful for the invention that do not rely on the direct analysis of ancestral haplotypes include, but are not limited to, sequencing of the DNA at one or more of the relevant positions; differential hybridisation of an oligonucleotide probe designed to hybridise at the relevant positions of the desired sequence; denaturing gel electrophoresis following digestion with an appropriate restriction enzyme, preferably following amplification of the relevant DNA regions; S1 nuclease sequence analysis; non-denaturing gel electrophoresis, preferably following amplification of the relevant DNA regions; conventional RFLP (restriction fragment length polymorphism) assays; selective DNA amplification using oligonucleotides which are matched for the wild-type sequence and unmatched for the mutant sequence or vice versa; or the selective introduction of a restriction site using a PCR (or similar) primer matched for the wild-type or mutant genotype, followed by a restriction digest. As indicated above, the assay may be indirect, i.e. capable of detecting a polymorphism at another position or gene which is known to be linked to a polymorphism of the interest. The probes and primers may be fragments of DNA isolated from nature or may be synthetic.

Amplification of DNA may be achieved by the established PCR methods or by developments thereof or alternatives such as the ligase chain reaction, QB replicase and nucleic acid sequence-based amplification.

In one method, a pair of PCR primers are used which hybridise to one allele but not another. Whether amplified DNA is produced will then indicate which allele is present.

Another method employs similar PCR primers but, as well as hybridising to only one of the alleles, they introduce a restriction site which is not otherwise there in any known allele.

In an alternative method, following amplification the products are sequenced. Preferably the products are sequenced without subcloning such that if two different alleles are present in the individual being tested their presence can easily be identified. If the products are subcloned a suitable number of subclones would need to be sequenced to ensure that both alleles have been analysed.

In order to facilitate subsequent cloning of amplified sequences, primers may have restriction enzyme sites appended to their 5′ ends. Thus, all nucleotides of the oligonucleotide primers are derived from the gene sequence of interest or sequences adjacent to that gene except the few nucleotides necessary to form a restriction enzyme site. Such enzymes and sites are well known in the art. The primers themselves can be synthesized using techniques which are well known in the art. Generally, the primers can be made using synthesizing machines which are commercially available.

A non-denaturing gel may be used to detect differing lengths of fragments resulting from digestion with an appropriate restriction enzyme. The DNA is usually amplified before digestion, for example using the polymerase chain reaction (PCR) method and modifications thereof.

PCR techniques that utilize fluorescent dyes may also be used to detect the genetic locus of interest. These include, but are not limited to, the following five techniques.

i) Fluorescent dyes can be used to detect specific PCR amplified double stranded DNA product (e.g. ethidium bromide, or SYBR Green I).

ii) The 5′ nuclease (TaqMan) assay can be used which utilizes a specially constructed primer whose fluorescence is quenched until it is released by the nuclease activity of the Taq DNA polymerase during extension of the PCR product.

iii) Assays based on Molecular Beacon technology can be used which rely on a specially constructed oligonucleotide that when self-hybridized quenches fluorescence (fluorescent dye and quencher molecule are adjacent). Upon hybridization to a specific amplified PCR product, fluorescence is increased due to separation of the quencher from the fluorescent molecule.

iv) Assays based on Amplifluor (Intergen) technology can be used which utilize specially prepared primers, where again fluorescence is quenched due to self-hybridization. In this case, fluorescence is released during PCR amplification by extension through the primer sequence, which results in the separation of fluorescent and quencher molecules.

v) Assays that rely on an increase in fluorescence resonance energy transfer can be used which utilize two specially designed adjacent primers, which have different fluorochromes on their ends. When these primers anneal to a specific PCR amplified product, the two fluorochromes are brought together. The excitation of one fluorochrome results in an increase in fluorescence of the other fluorochrome. Such assays may also use a ligase so that the two annealed primers joined together.

### EXAMPLES

### Example 1

### Identification of Haplospecific Geometric Elements in Duplicated Genes Encoding Complement Control Proteins

### Methods

### Identification of Duplicons

The genomic region containing CR1, MCP-like, CR1-like and MCP at 1q32, was taken from the NCBI database (http://www.ncbi.nlm.nih.gov/) (position 1124945-1449694 on contig NT_{—}021877.16 (gi:37539616); accession numbers AL691452.10, AL137789.11, AL365178.10 and AL035209.1). This sequence was compared against itself using Dotter (Sonnhammer and Durbin, 1995) to identify evidence of duplication (McLure et al. 2005a).

### Selection of Primer Sites Present in all Duplicons

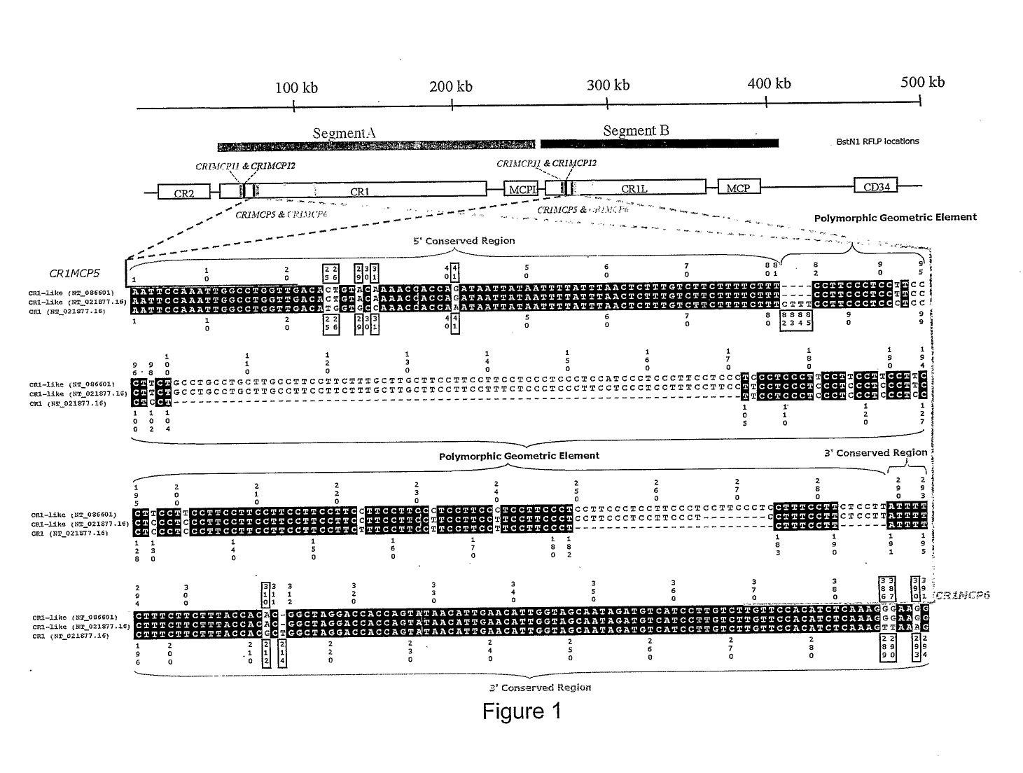

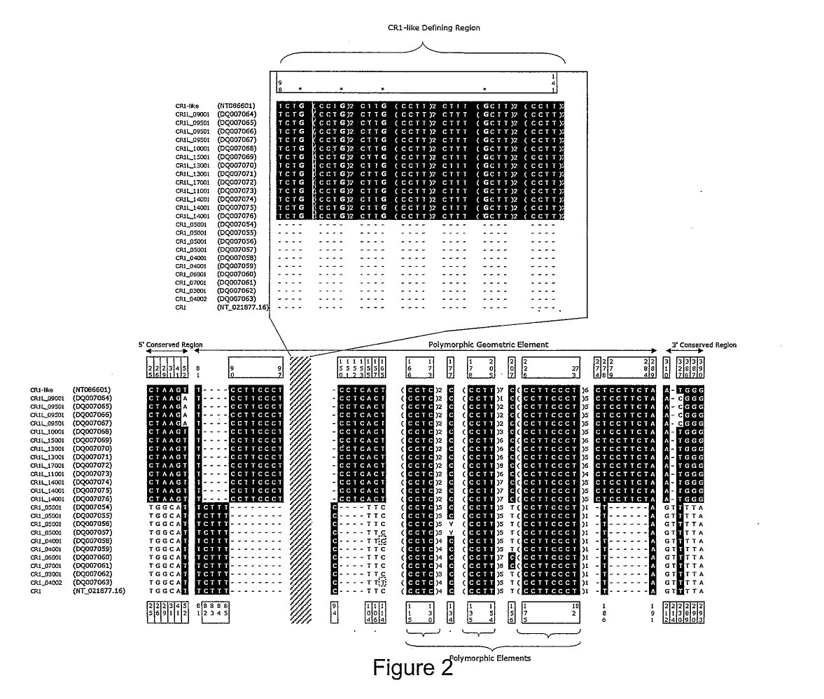

Segment A, containing CR1 and MCP-like was compared to Segment B, containing CR1-like and MCP. Regions within these two segments which shared a complex geometric element were identified as targets (McLure et al. 2005a). The geometric element must vary in size between the duplicates (see FIGS. 1 and 2) but also contain enough homology either side of the element so as to enable the design of primers that will bind and amplify within each segment. The resulting mix of products has the potential to define extensive haplotypes.

Duplicons at position 1150081-1150372 (CR1) and 1322386-1322768 (CR4-like) of NT_{—}021877.16 were aligned using Clustalw (http://www.es.embnet.org/cgi-bin/clustalw.cgi). Using Primer3 (http://frodo.wi.mit.edu/cgi-bin/primer3/primer3 www.cgi), primers were designed so that a single primer pair will bind and amplify both duplicates or even more if, as expected, there are more than two duplicated segments on some haplotypes.

Primer sequences were compared to the NCBI databases using BLASTN (http://www.ncbi.nlm.nih.gov/BLAST/) at low stringency. Sequence identities which matched the primers in both the forward and reverse directions were identified. The only significant matches for primers in question were in close proximity and it could therefore be assumed the primer pair would amplify within a polymorphic frozen block (PFB). Analysis of the amplified elements with matches from the Celera database (NT_{—}086601 position 1267344-1267734) suggests the duplicated elements are polymorphic between individuals (FIG. 1). The intention is to amplify as many duplicated sites as possible so long as there is no amplification of unlinked sequences. In the case of the RCA complex, there is a risk of interference from unlinked priming because CCPs are widely distributed. Accordingly, we used a three generation nuclear family to test the selected primers. If the primers are valid, segregation through generations should be apparent.

Comparison of Products within 3 Generation Families

Families with disputed paternity were avoided. Individuals were compared as blind pairs. Amplicon peaks were numbered successively.

### Assignment of Haplotypes

Once the profiles of individual subjects were defined and compared, the data were interpreted within the context of the family structure. For example, the grandfather is designated ab and the grandmother cd. Next, the second generation, designated II, is inspected to determine which part of the parental profiles were transmitted. In this way a, b, c, and d haplotypes can be deduced. As a test of the validity of these assignments, the next generation (III) is examined. Haplotypic profiles from generation I should be retained even when they are associated with haplotypes not present in the previous two generations.

Determination of Population Frequencies with Comparison of Functions and Diseases

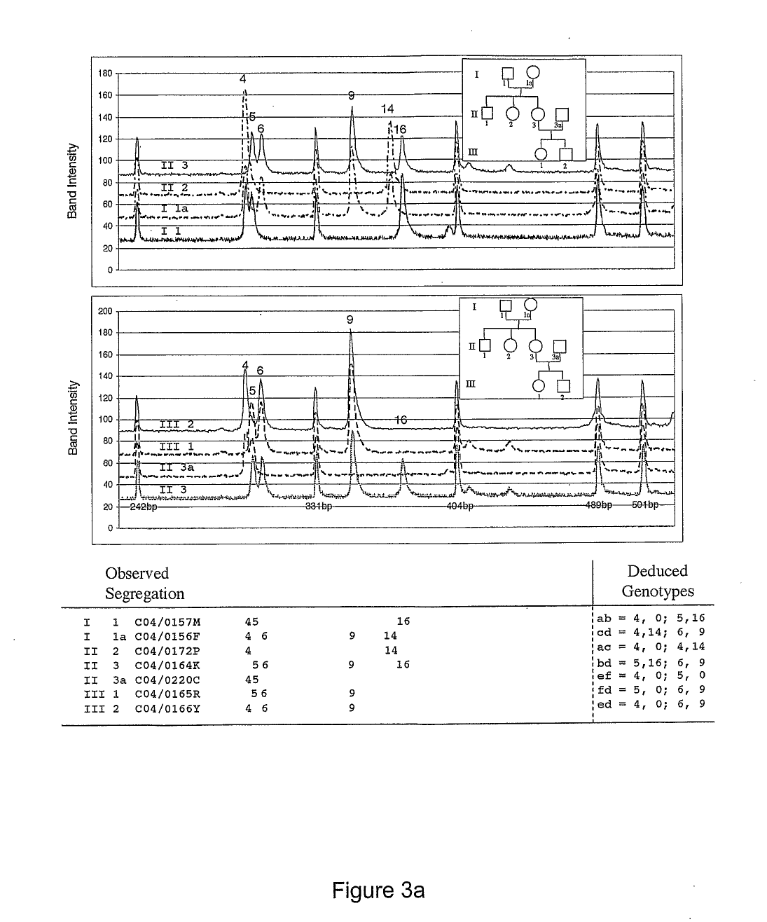

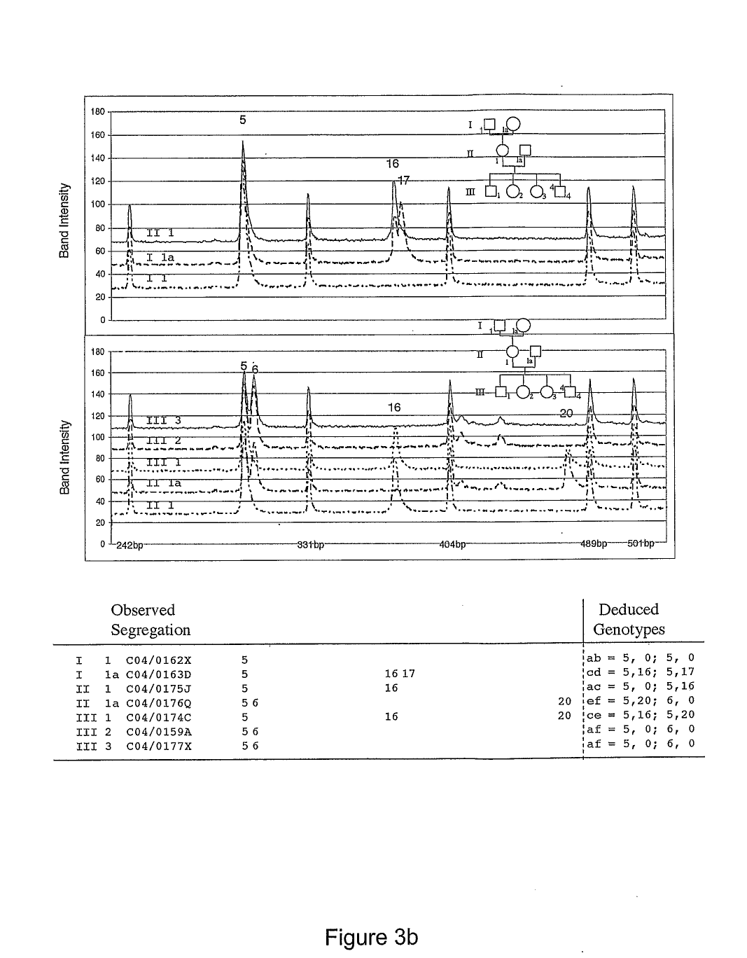

Haplotypic profiles verified by family studies were given a number here referred to as 01, 02 . . . 99 (see Table 1). These profiles can then be recognised in other families and in other homozygotes. Having defined common ancesteral haplotypes, we then examine heterozygotes to determine if 2 assigned haplotypes are present. Product intensity is also considered as illustrated in FIG. 3. We use the Hardy Weinberg test as an indication of the validity of assignments. Population and disease studies are then justified.

TABLE 1
RCA haplotypes in an ethnically diverse DNA panel.

| Ancestral | GMT typing | | BstN1 | Frequency | |
|---|---|---|---|---|---|
| Haplotype | P5 + 6 | P11 + 12 | typing | n | % |
| 01 | 5.0 | 1.13 | G | 156-165 | 24-25 |
| 02 | 4.0 | 1.13 | G | 80-83 | 12-13 |
| 03 | 5.16 | 1.15 | G | 75-77 | 12 |
| 04 | 5.13 | 1.11 | G | 18-24 | 3-4 |
| 05 | 6.0 | 4.13 | T | 24-29 | 4 |
| 06 | 5.14 | 1.15 | G | 18 | 3 |
| 07 | 5.17 | 1.15 | G | 16 | 2 |
| 08 | 6.13 | 5.11 | T | 11-16 | 2 |
| 09 | 5.15 | 1.15 | G | 15 | 2 |
| 10 | 6.0 | 1.13 | T | 12-13 | 2 |
| 11 | 6.9 | 4.17 | G | 7-9 | 1 |
| 12 | 4.0 | 1.12 | G | 8 | 1 |
| 13 | 5.0 | 1.19 | G | 6-8 | 1 |
| 14 | 5.0 | 1.18 | G | 7-8 | 1 |
| 15 | 4.14 | 1.11 | G | 8 | 1 |

| | | |
|---|---|---|
| Sub-Total | 461-497 | 71-77 |
| Other | 108-152 | 17-23 |
| Total | 569-649 | 88-100 |

The P5 + 6 haplotypes identified in the segregation studies and homozygotes were used to deduce the haplotypes of additional unrelated individuals. A similar approach was taken with P11 + 12 and the combination of P5 + 6 and P11 + 12 used to assign the Ancestral Haplotype number. No deviation from Hardy-Weinberg equilibrium was observed confirming that heterozygotes can be assigned. Only the 15 most common are shown here. These account for approximately 70% of the population studied. After assignment of these, BstN1 typing revealed that each had either G or T at the cutting site on CR1. At least 15 rarer haplotypes were identified but at a frequency of less than 1%. Some of these may be ethnic specific. Some haplotypes also differ in minor bands not illustrated here.

The inventors also generated all theoretically possible haplotypes from the alleles found in each subject. Those occurring in more than 3 subjects were considered further. In some cases, the frequencies were similar to those shown in Table 1 but there were major differences. Some of the common theoretically possible haplotypes were not observed as homozygotes and were not assigned.

### Primer Sequences

P5 + 6

(SEQ ID NO: 1)

| | |
|---|---|
| CR1MCP5 | 5′AAT TCC AAA TTG GCC TGG TTG A 3′ |
| and | |

(SEQ ID NO: 2)

| | |
|---|---|
| CR1MCP6 | 5′CCT TCC CTT TGA GAT GTG GAA CA 3′. |
| P11 + 12 | |

(SEQ ID NO: 3)

| | |
|---|---|
| CR1MCP11 | 5′ GTC AGC TTG GAT TGC CCT TGG TTC TA 3′ |
| and | |

(SEQ ID NO: 4)

| | |
|---|---|
| CR1MCP12 | 5′ CCT GGG CAA CAA AGC AAG ACA TTG T 3′. |

### Polymerase Chain Reaction

Genomic DNA was prepared using the standard salting-out method.

PCR reactions were performed in a 96-well Palm Cycler (Corbett Research) in 20 μl volumes using 100 ng of template DNA, 1.3 U Taq Polymerase (Fisher Biotec), 10 μmol of the forward and reverse CR1MCP primers, 200 μM of each dNTP, 2 mM MgCl₂ and 1×PCR buffer (Fisher Biotec). The samples were denatured at 94° C. for 5 min, followed by 30 cycles each comprising 30 seconds at 94° C., 45 seconds at 58° C. and 45 seconds at 72° C. The last cycle was followed by an additional extension for 5 minutes at 72° C.

### Detection of Amplicons and Haplotypes

The separation and detection of the allelic variants of CR1 and CR1-like was done with the Corbett Research GS-3000 automated gel analysis system. One microlitre of PCR product was mixed with 1 μl of loading buffer containing Puc19 molecular weight ladder. One microlitre of the PCR sample and loading buffer mixture was then added to a 32 cm long, 48 well, 4% polyacrylamide, ultra-thin gel and pulsed for 10 seconds. Excess sample was then flushed and the gel was run at 2000 V for 180 minutes.

### Gel Analysis and Profile Generation

The gel image was analysed using BioRad Quantity One gel analysis software. Lanes were defined, amplicons detected and standards assigned. Densimetric profiles were generated and lanes were aligned using the internal pUC19/Hpa II (Fisher Biotec) standards.

### CR1 and CR1-Like Sequencing

The amplification primers used were:

CR1 specific primers -

(SEQ ID NO: 9)

| | |
|---|---|
| CR1-F1: | 5′ AAT TCC AAA TTG GCC TGG TT 3′ |
| and | |

(SEQ ID NO: 10)

| | |
|---|---|
| CR1-R1: | 5′ AAA CTTT AAC TTT GAG ATG TGG AAC A 3′ |

CR1-like specific primers -

(SEQ.ID NO: 1)

| | |
|---|---|
| CR1MCP5: | 5′ AAT TCC AAA TTG GCC TGG TTG A 3′ |
| and | |

(SEQ ID NO: 2)

| | |
|---|---|
| CR1MCP6: | 5′ CCT TCC CTT TGA GAT GTG GAA CA 3′. |

PCR products were analysed using a 2% agarose gel. Individual bands were cut from the gel and purified using Amersham Biosciences GFX PCR Gel Band Purification Kit. The purified products were amplified as above and sequenced.

### BstN1 Digestion

Polymorphism at nucleotide 3093 was detected using PCR amplification and BstN1 digestion. This was performed using primers and methods detailed by Birmingham (Birmingham et al. 2003). PCR conditions were as above, except the annealing step was at 60° C. for 45 seconds. Sequence analysis suggest that the primers amplify the site telomeric of CR1 j1 (repeated in CR1 as shown in FIG. 1) but not CR1-like because of differences in the primer sites.

### Results

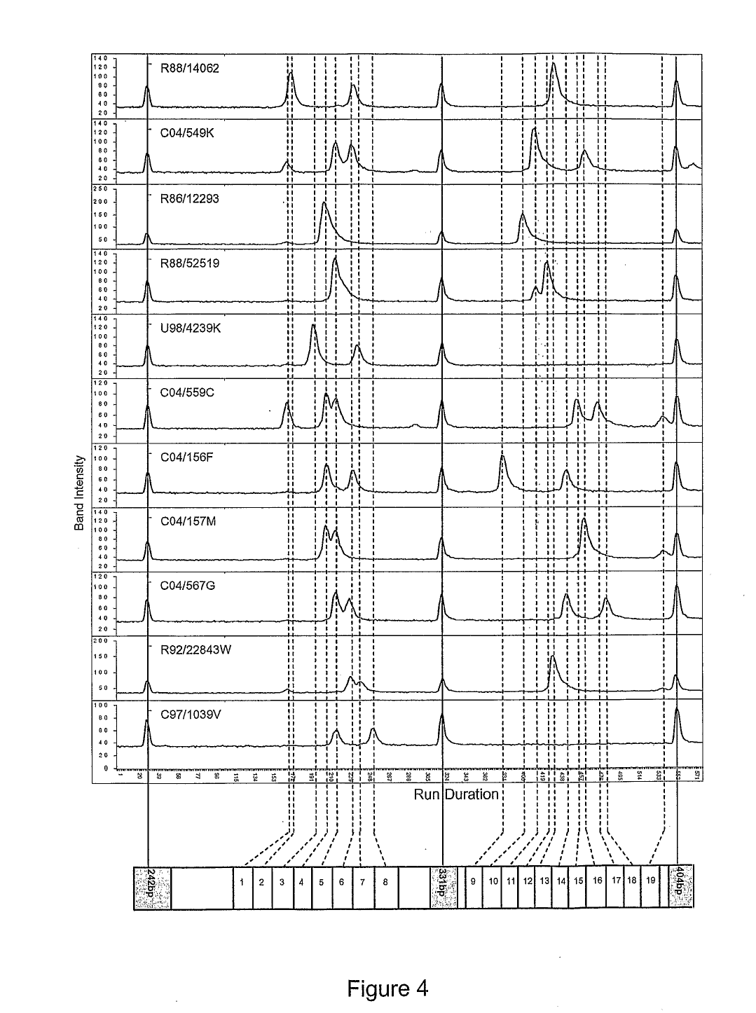

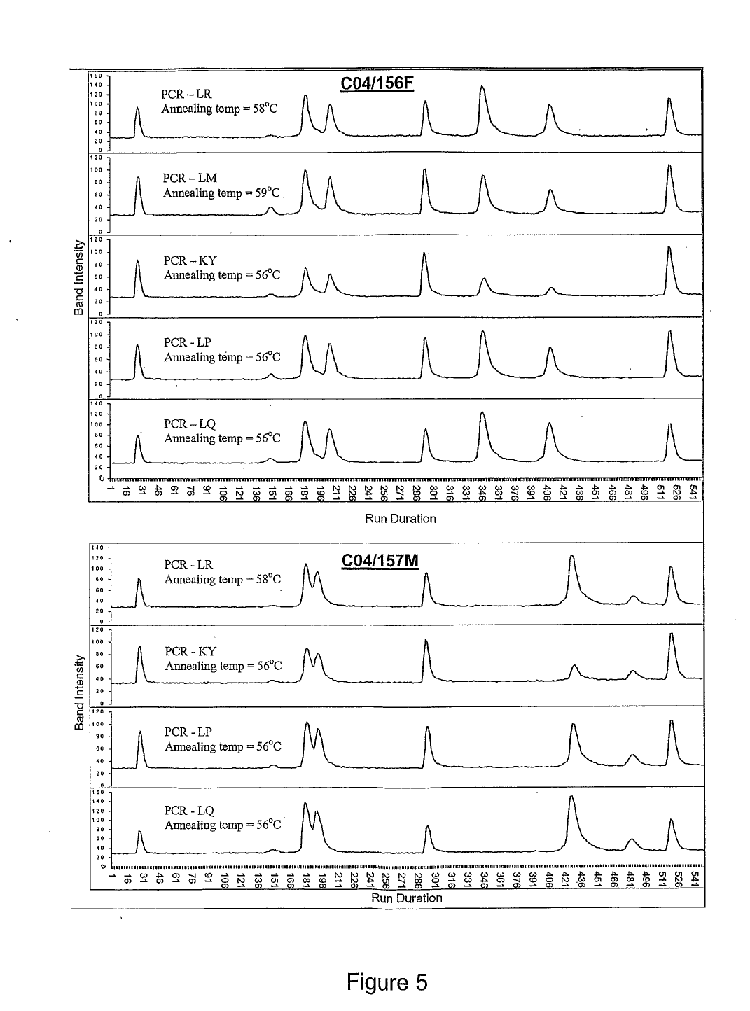

The present inventors have identified extensive segmental duplication involving Complement Receptor 1 (CR1) and Membrane Cofactor Protein (MCP) (FIG. 1). With primers P5+6 designed to amplify at duplicated sites separated by hundreds of kilobases, the inventors observed multiple diverse products in a screening panel of 60 human subjects selected to include the major ethnic groups and some relevant diseases. As shown in FIG. 4, there are 1, 2 or 3 products in the range around 300 bp and 0, 1, 2 or 3 products in the range around 350 bp. Each of the 11 subjects has a unique composite profile. As shown in FIG. 5, these are highly reproducible with only minor differences under different conditions of amplification.

The inventors then studied 3 generation families in order to determine whether combinations of products define transmissible haplotypes. The families had already undergone MHC typing which was consistent with stated parentage. In all cases, the RCA haplotypes were unequivocal and faithfully transmitted. For example, as shown in FIG. 3, each product can be numbered according to length such that I1 in family 1 has the 4, 5 and 16 profile which resolves through segregation analysis into two haplotypes (a=4 with null and b=5 with 16) and therefore the genotype 4,0;5,16. Note also that in II 2 (ac), the intensity of product 4 is increased in keeping with the genotype 4,0;4,14 and homozygosity of 4. Similarly, in Family 2, I1 (ab) is homozygous for 5.

In spite of some homozygosity, there is extreme polymorphism as illustrated by the fact that there are 11 different profiles and genotypes in the 12 subjects. In each family there are 3 unrelated individuals (ab,cd,ef). In these 6 subjects there are 9 different haplotypes. In the case of the 4,0 and 5,0 haplotypes the frequencies were 2/12 and 3/12 respectively suggesting that these may be relatively common and functionally important ancestral haplotypes. We therefore reviewed the profiles of the panel of 60 subjects and found that most haplotypes could be assigned using the iterative strategies described in the methods.

Confirmation of these assignments was obtained by amplifying other duplicated sequences with primers 11 and 12 shown in FIG. 1 and by determining the presence or absence of the BstN1 (G3093T) cutting site (Birmingham et al. 2003) on different haplotypes (Table 1). These results demonstrated that the haplotypes contain haplospecific features at multiple sites. For example 02 contains 4,0 with P5+6, and 1, 13 with P11+12 and is G3093 whereas 08 is P5+6=6,13 and P11+12=5,11 and is G3093T.

The inventors then tested a separate panel of 322 subjects. The frequencies of haplotypes in this dataset are as expected from the 2 smaller panels and are shown in Table 1 which also proposes designations for the more common ancestral haplotypes.

To characterise the haplotypes in more detail we sequenced representative P5+6 products. Based on the available genomic sequences, we expected that the products of less than 331 bp would be from CR1 and those above 331 would be from CR1-like (FIG. 4). We therefore established operational criteria for assignment using the patterns shown in FIG. 2. All sequences were as expected.

As shown in Table 1 and FIG. 1, some haplotypes fail to generate a CR1-like product when amplified with P5+6. Since P11+12 yield 2 products per haplotype we conclude that there is a further polymorphism, probably an indel, which negates amplification with P5+6 on the CR1-like null haplotypes. Of further interest, the data suggest that some haplotypes contain more than 2 duplicons. In fact, on longer gels there are additional products which have not been shown here.

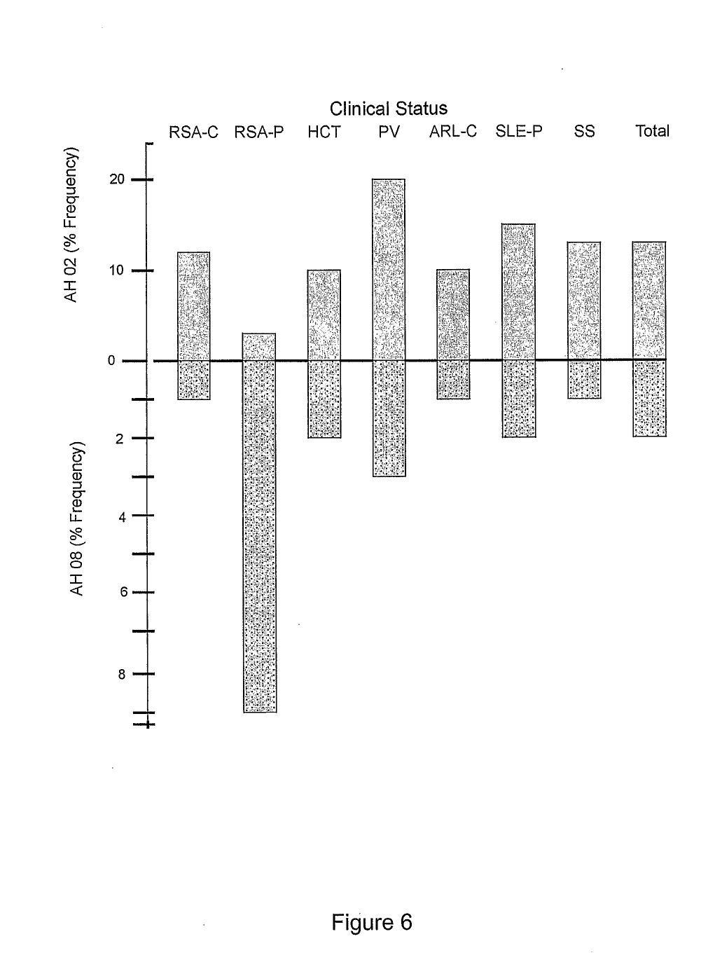

In Table 2 we show the frequencies in the panel of 322 arranged by clinical subset. The distribution of CR1-01 is similar in all groups but CR1-02 is rare in patients with RSA and frequent in those with Psoriasis Vulgaris (PV) (FIG. 6). The reverse is seen with CR1-04 and -08. Indeed when haplotypes are compared in terms of RSA-P v PV the ratios vary tenfold. Note also that more than 50% of ha plotypes are yet to be defined in RSA-P whereas the corresponding figure in PV is 10-19%.

These results provide the first evidence for a role of the RCA complex in RSA.

The present study shows of the utility of the GMT approach. This simple procedure has demonstrated linked polymorphisms including at least one of functional significance (Birmingham et al. 2003). Short of sequencing and somehow assembling hundreds of kilobases in at least 30 subjects, we know of no other approach which could reveal more than 20 different haplotypes with such extensive polymorphism. The rationale for the assay is that sequence polymorphism is concentrated in some regions or quanta, which, in our experience, are also rich in duplications. We recommend the use of larger segments with major indels and therefore differences in length when the 2 or more copies are compared.

Insertions and deletions (indels) are also associated with concentrations of polymorphism (Longman-Jacobsen et al. 2003). These indels are often complex and degenerate suggesting a mechanism for divergence between the different duplicons. As described in FIG. 1, the sequence amplified includes an L1 (LIM5 or L1P4) which must have anteceded the duplication but which is different when the 2 copies are compared. There are also differences in the 5′ sequence but most of the variations in length are due to the very complex TC rich region which we refer to as a Polymorphic or Haplospecific Geometric Element (HGE). This contrasts with a microsatellite in that there are diverse units of different lengths and yet the sequences have a geometric pattern (FIG. 2). Other features we associate with such HGEs are stability, complementary sequences, uniqueness within the genome and extreme polymorphism. A study using microsatellites in the vicinity of CR1 revealed little polymorphism but did suggest that there is limited recombination as predicted by the PFB hypothesis (Heine-Suner et al. 1997).

TABLE 2
Percentage frequencies of ancestral haplotypes
in different clinical groups.

| An- | | | | | | | |
|---|---|---|---|---|---|---|---|
| ces- | | | | | | | |
| tral | Disease group | | | | | | |
| Hap- | RSA-C | RSA-P | HCT | PV | ARL-C | SLE-P | SS |
| lo- | Number of chromosomes in sample | | | | | | |
| type | n = 74 | n = 92 | n = 48 | n = 132 | n = 84 | n = 58 | n = 156 |
| | Haplotype Frequencies (%) | | | | | | |
| 01 | 15 | 17-18 | 25 | 23-25 | 22-24 | 21-24 | 32 |
| 02 | 12 | 2-3 | 10 | 20 | 9-10 | 13-15 | 13 |
| 03 | 11 | 6-7 | 17 | 7 | 11-12 | 8 | 18 |
| 04 | 3 | 3-5 | 2 | 2-3 | 3 | 3 | 3-4 |
| 05 | 8 | 2 | 6 | 4-7 | 2-3 | 3 | 2 |
| 06 | | | 2 | 1 | 3 | 5 | 6 |
| 07 | | | 6 | 3 | 3 | 2 | 3 |
| 08 | 1 | 6-9 | 2 | 3 | 1 | 2-3 | 1 |
| 09 | | 1 | 2 | 4 | 2 | 5 | 1 |
| 10 | 1-3 | 2 | 2 | 4 | 1 | | 1 |
| 11 | | | 2 | 1-2 | 2 | 2 | 1 |
| 12 | | | 4 | 1 | 3 | | 1 |
| 13 | 3 | | | 1 | | 2 | 1-2 |
| 14 | 1 | 2-3 | 2 | | | 3 | 1 |
| 15 | 3 | 1 | | 1 | 1 | | 2 |
| Other | 42-43 | 51-59 | 17 | 10-19 | 29-34 | 26-32 | 15-17 |
| | Number of possible haplotypes | | | | | | |
| | n = 76 | n = 102 | n = 48 | n = 142 | n = 92 | n = 62 | n = 160 |

Abbreviations as in legend for FIG. 6. The n value refers to the number of Chromosomes and adds to 644. Because of some ambiguities, ranges of frequency are shown in some instances and the total number of possible haplotypes is 682. The percent frequencies are similar in the two control groups and in HCT, SLE and SS but some haplotypes are strikingly different when RSA-P and PV are compared.

PFBs are remarkable since, although they contain extreme polymorphism, duplicons and indels, they behave as though they become frozen after which they appear to be resistant to recombination and mutation. In terms of calculations of linkage disequilibrium, higher values are found within, rather than between PFB, but cannot be expected when haplotypes share common alleles in different combinations.

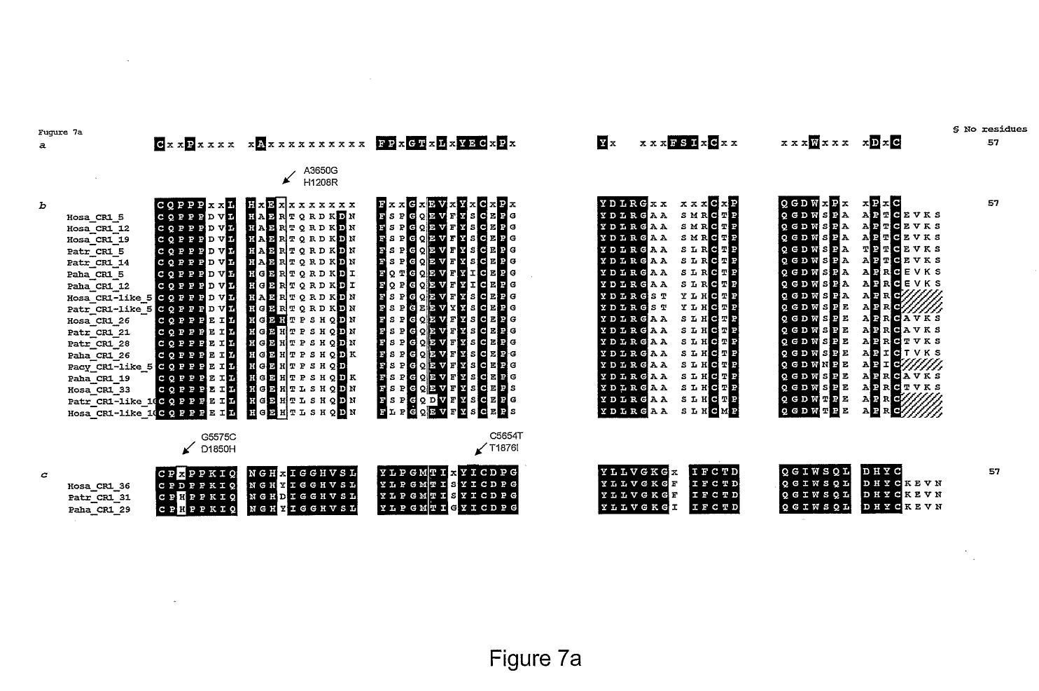

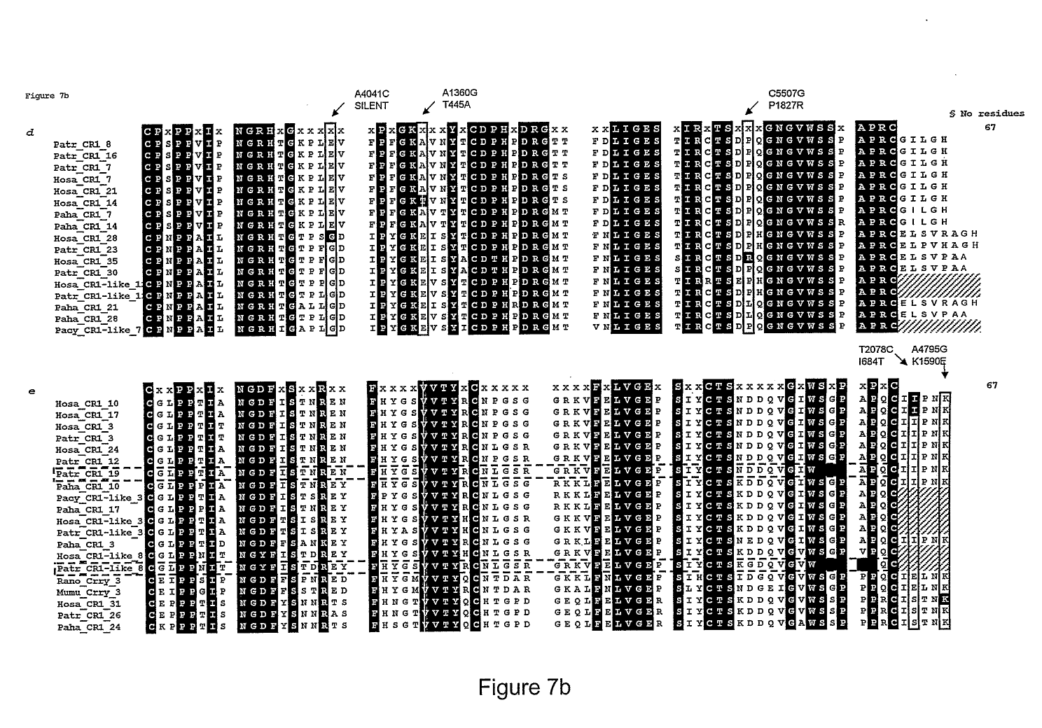

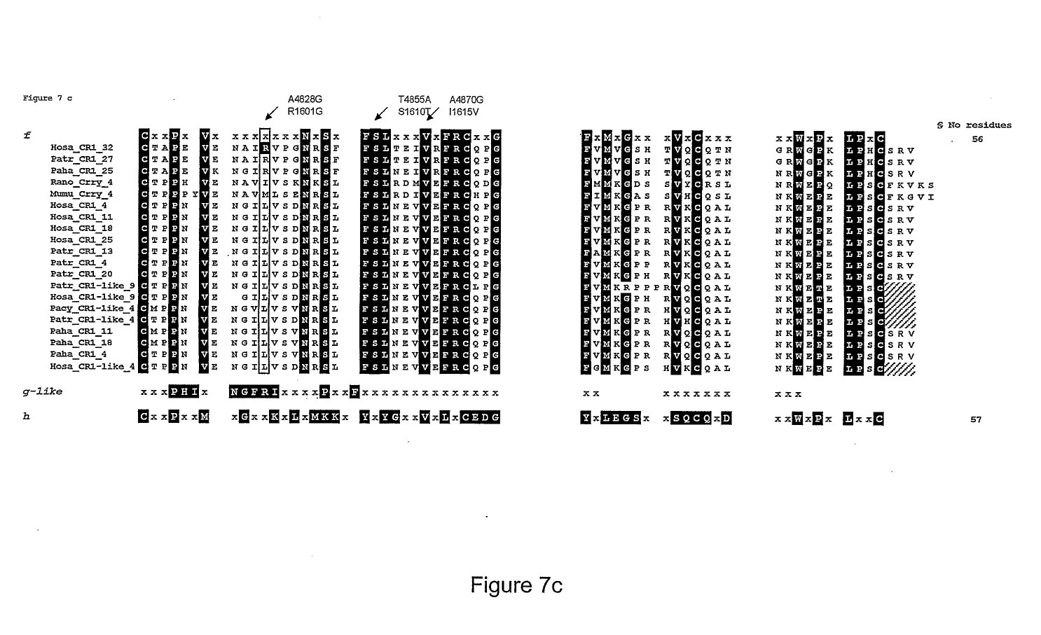

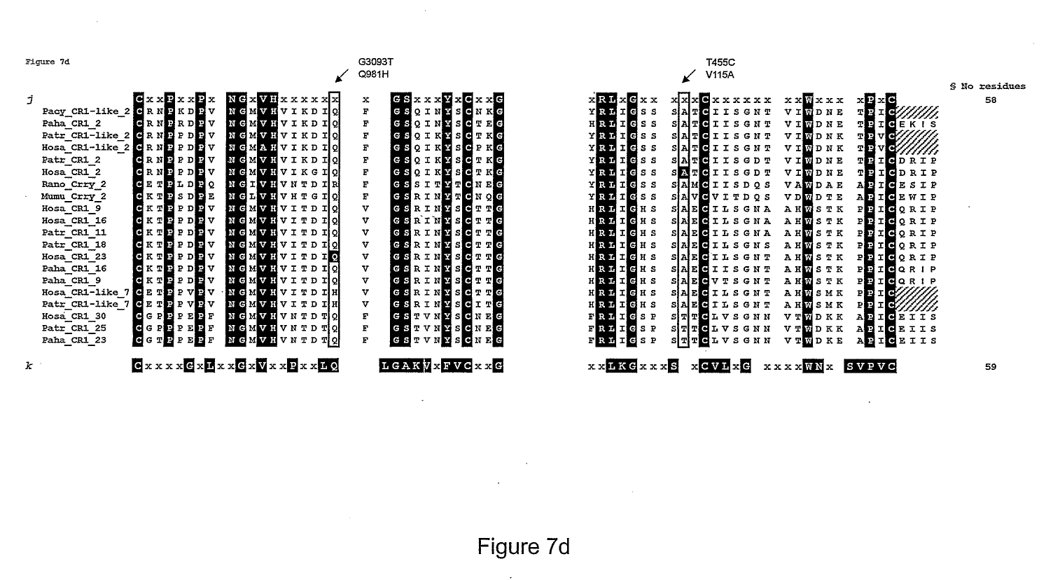

The alternative sequences within a PFB (ancestral haplotypes) are inherited faithfully over many generations. In the MHC, ancestral haplotypes which are now found in tens of millions of the population have proven, when sampled, to be identical at the sequence level. We expect that the same will be true of CCP region and that these conserved polymorphisms will be critical in explaining differences in function and disease (see FIG. 7). Included in the possibilities are inflammatory diseases such as RSA, SLE and SS and differences in susceptibility to viruses, such as measles, which exploit CCPs, such as MCP, as receptors.

### Example 2

### Identification of Ancestral Haplotypes Significantly Decreased in Indian Samples from RSA Patients

Regression analyses was performed using WinBugs (V1.4.1 http://www.mrc-bsu.cam.ac.uk/bugs/winbugs/contents.shtml) which uses Bayesian MCMC methods to estimate empirical 95% credible intervals (CI), which are less biased for small sample sizes. The odds ratio is significant with a p-value <0.05 if these 95% credible intervals do not include 1. The analyses were performed with the assistance of an Excel-Winbugs interface Add-in BugsXLA (v2.1, Phil Woodward http://www.pipshome.freeserve.co.uk/stats/). As is customary when there are zero cell counts, a constant of 0.5 was added to all cells counts as odds ratios are not defined in these instances.

Indian samples (RSA samples pooled) were compared to Caucasian samples (pooled over 5 groups). The results are provided in Table 3.

A number of the AH's are significantly decreased in Indian samples compared to Caucasians.

### Example 3

### CR1 Haplotype Analysis of Recurrent Spontaneous Abortion Patients

Analysis was performed as described above in relation to Example 2. The results are provided in Table 4.

TABLE 3
Indian samples (RSA samples pooled)
were compared to Caucasian samples.

| | GMT TYPING | | INDIANS vs CAUCASIANS |
|---|---|---|---|
| P5 + 6 | P11 + 12 | Haplotype | Odds Ratio (95% CI) |
| 5.0 | 1.13 | H1 | 0.28 (0.17, 0.46) |
| 4.0 | 1.13 | H2 | 0.15 (0.07, 0.31) |
| 5.16 | 1.15 | H3 | 0.31 (0.16, 0.57) |
| 5.13 | 1.11 | H4 | 0.94 (0.24, 3.53) |
| 6.0 | 4.13 | H5 | 0.54 (0.19, 1.45) |
| 5.14 | 1.15 | H6 | 0.01 (0.00, 0.24) |
| 5.17 | 1.15 | H7 | 0.01 (0.00, 0.22) |
| 6.13 | 5.11 | H8 | 1.17 (0.42, 3.28) |
| 5.15 | 1.15 | H9 | 0.09 (0.01, 0.46) |
| 6.0 | 1.13 | H10 | 0.30 (0.03, 2.05) |
| 6.9 | 4.17 | H11 | 0.14 (0.01, 0.74) |
| 4.0 | 1.12 | H12 | 0.02 (0.00, 0.43) |
| 5.0 | 1.19 | H13 | 0.39 (0.07, 1.74) |
| 5.0 | 1.18 | H14 | 1.18 (0.29, 4.93) |
| 4.14 | 1.11 | H15 | 0.47 (0.08, 2.22) |
| Other | Other | Other | 1 |

p_{exact} = 0.000002

TABLE 4
Analysis of recurrent spontaneous abortion patients
(RSA-P) compared with a control group (RSA-C)

| | |
|---|---|
| GMT TYPING | RSA-P vs RSA-C |

| P5 + 6 | P11 + 12 | Haplotype | Odds Ratio (95% CI) |
|---|---|---|---|
| 5.0 | 1.13 | H1 | 0.91 (0.40, 2.06) |
| 4.0 | 1.13 | H2 | 0.08 (0.01, 0.47) |
| 5.16 | 1.15 | H3 | 0.64 (0.21, 1.88) |
| 5.13 | 1.11 | H4 | 1.83 (0.24, 22.40) |
| 6.0 | 4.13 | H5 | 0.13 (0.01, 0.85) |
| 6.13 | 5.11 | H8 | 4.32 (0.72, 47.80) |
| 5.15 | 1.15 | H9 | 4.68 (0.10, 1815) |
| 6.0 | 1.13 | H10 | 4.69 (0.11, 1542) |
| 6.9 | 4.17 | H11 | 0.09 (0.00, 3.78) |
| 5.0 | 1.19 | H13 | 0.04 (0.00, 1.38) |
| 5.0 | 1.18 | H14 | 1.83 (0.24, 22.49) |
| 4.14 | 1.11 | H15 | 0.04 (0.00, 1.36) |
| Other | Other | Other | 1 |

p_{exact} = 0.006

Haplotype 2 is significantly decreased in recurrent spontaneous abortion patients and may be protective of RSA.

The odds ratio for haplotype 8 is not significant, but it is difficult for the present analysis to detect low frequency haplotypes as significantly different. This haplotype however probably contributes substantially to the overall p-value indicating the frequency is different between the two groups. The analysis on a collapsed table with just the higher frequency haplotypes (H1, H2, H3 & All Other) gives a p-value of 0.04-still significant, but not as striking. We attribute the difference to haplotype 8. However, with a frequency of 7% in the RSA-P group, it is unlikely to be a major RSA genetic susceptibility factor.

### Example 4

### CR1 haplotype analysis of Haemochromatosis (HCT), Psoriasis Vulgaris (PV), Systemic Lupus Erythematosus (SLE) and Sjögren's Syndrome (SS) Patients

Analysis was performed as described above in relation to Example 2. The results are provided in Table 5.

There is evidence that H1 and H2 are increased in PV and H1 and H3 are increased in SS. Analysis on a collapsed table with just the higher frequency haplotypes (H1, H2, H3 & All Other) provided a p-value for PV vs controls of 0.11 and for SS vs controls of 0.06.

TABLE 5
Analysis of Haemochromatosis (HCT), Psoriasis Vulgaris (PV), Systemic Lupus Erythematosus
(SLE) and Sjögren's Syndrome (SS) patients with a control group.

| GMT TYPING | | | HCT vs CONTROLS | | PV vs CONTROLS | | SLE vs CONTROLS | | SS vs CONTROLS | |
|---|---|---|---|---|---|---|---|---|---|---|
| P5 + 6 | P11 + 12 | Haplotype | Odds Ratio (95% CI) | | Odds Ratio (95% CI) | | Odds Ratio (95% CI) | | Odds Ratio (95% CI) | |
| 5.0 | 1.13 | H1 | 2.01 | (0.72, 5.66) | 2.39 | (1.06, 5.30) | 1.22 | (0.49, 3.00) | 3.08 | (1.43, 6.55) |
| 4.0 | 1.13 | H2 | 1.44 | (0.38, 5.19) | 3.55 | (1.40, 9.43) | 1.63 | (0.56, 4.71) | 1.94 | (0.75, 5.18) |
| 5.16 | 1.15 | H3 | 2.10 | (0.63, 6.99) | 1.25 | (0.43, 3.59) | 0.74 | (0.21, 2.45) | 2.67 | (1.09, 6.58) |
| 5.13 | 1.11 | H4 | 0.39 | (0.00, 18.27) | 0.19 | (0.00, 8.63) | 1.61 | (0.10, 27.85) | 3.12 | (0.39, 39.81) |
| 6.0 | 4.13 | H5 | 2.86 | (0.35, 24.24) | 4.03 | (0.85, 24.51) | 0.11 | (0.00, 3.32) | 1.60 | (0.26, 11.05) |
| 5.14 | 1.15 | H6 | 0.13 | (0.00, 3.70) | 0.91 | (0.13, 5.68) | 0.56 | (0.04, 4.52) | 2.87 | (0.73, 13.05) |
| 5.17 | 1.15 | H7 | 2.85 | (0.48, 16.91) | 1.79 | (0.37, 8.87) | 0.55 | (0.04, 4.46) | 1.41 | (0.29, 6.94) |
| 6.13 | 5.11 | H8 | 2.83 | (0.17, 43.21) | 2.58 | (0.27, 32.49) | 3.07 | (0.31, 37.68) | 2.05 | (0.21, 24.63) |
| 5.15 | 1.15 | H9 | 1.00 | (0.07, 8.67) | 2.75 | (0.64, 13.32) | 1.64 | (0.29, 9.03) | 0.38 | (0.03, 2.98) |
| 6.0 | 1.13 | H10 | 21.03 | (0.44, 6298) | 28.99 | (1.12, 7317) | 1.59 | (0.00, 844) | 1.09 | (0.00, 566) |
| 6.9 | 4.17 | H11 | 1.47 | (0.11, 14.98) | 2.02 | (0.32, 13.83) | 0.82 | (0.06, 8.07) | 1.06 | (0.14, 8.01) |
| 4.0 | 1.12 | H12 | 1.94 | (0.26, 13.08) | 0.47 | (0.04, 3.78) | 0.08 | (0.00, 2.07) | 0.73 | (0.11, 4.55) |
| 5.0 | 1.19 | H13 | 2.81 | (0.00, 1857) | 19.26 | (0.63, 5814) | 11.70 | (0.26, 4064) | 22.76 | (0.87, 6575) |
| 5.0 | 1.18 | H14 | 17.57 | (0.45, 2852) | 1.13 | (0.00, 397) | 19.53 | (0.76, 3014) | 6.60 | (0.18, 1121) |
| 4.14 | 1.11 | H15 | 0.39 | (0.00, 18.77) | 1.35 | (0.08, 23.50) | 0.22 | (0.00, 10.20) | 3.14 | (0.39, 43.03) |

| | | | | | | |
|---|---|---|---|---|---|---|
| Other | Other | Other | 1 | 1 | 1 | 1 |
| | | | p_{exact} = 0.80 | p_{exact} = 0.17 | p_{exact} = 0.66 | p_{exact} = 0.20 |

Overall p_{exact} = 0.09 (over 5 groups)

### Example 5

### Epistatic Interaction Between the MHC and the Regulators of Complement Activation (RCA) Complex in Primary Sjögren's Syndrome

### Materials and Methods

### Study Participants

Ninety eight population based Caucasian controls and 115 Caucasian pSS patients from the South Australian Sjögren's Syndrome research registry were included in the study. All patients met the revised 2002 American-European consensus research classification criteria for pSS (Vitali et al. 2002). Anti-Ro/La autoantibody specificity was determined by ELISA (Immunoconcepts RELISA) using recombinant Ro60 and La proteins, as part of standard diagnostic procedure. Sera from patients with anti-La were further tested by CIEP (Beer et al. 1996) to confirm whether or not anti-La antibodies detected by ELISA were able to be detected by this method. HLA typing of pSS patients (serological class I and molecular class II) was performed by the Transplantation Laboratory, Australian Red Cross Blood Service, SA Division. The study was approved by the Human Ethics Committee of The Queen Elizabeth and Royal Adelaide Hospitals and all patients gave informed, written consent.

### CR1 Haplotyping

CR1 haplotyping was performed by the GMT technique as previously described in Example 1. Briefly, two separate PCR reactions using primer sets CR1MCP5&6 and CR1MCP11&12 were performed on each genomic DNA sample. The primers sets were each designed to amplify a complex geometric element common to both duplicated segments in the CR1 region (Segment A containing CR1 and MCP-Like and Segment B containing CR1-Like and MCP), resulting in a mix of PCR products of different sizes that defines CR1 haplotypic variation. The PCR products were separated on the basis of size on a Corbett Research GS-3000 automated gel analysis system. Haplotype assignment and nomenclature was as previously described in Example 1.

### Statistical Analysis

Contingency table analysis of CR1 genotype and haplotype frequencies was performed by χ2 analysis, using the log-likelihood ratio χ2 statistic. Significant associations were further reported as odds ratios (OR) with 95% confidence intervals (CI).

### Results

### CR1 Haplotype Diversity

More than 20 haplotypes have been defined, although the majority are rare. In the current study of 213 Caucasians (pSS and controls combined), there were 3 relatively common haplotypes (Ancestral Haplotypes AH1, AH2 and AH3 as designated in Example 1) each with a frequency of >10%. These three haplotypes combined accounted for 56% of the total haplotypes in the sample. There were a further 14 haplotypes with a frequency between 1-3%. These frequencies were considered too low to be informative given the study sample sizes and were therefore combined for analysis purposes.

### CR1 Haplotype Frequencies in pSS vs Controls

CR1 haplotype frequencies were significantly different between pSS patients and controls (χ²=15.5, df=3, p=0.001, Table 6). Both AH1 (OR 2.2 (1.4,3.6) and AH3 (OR 2.6 (1.3,5.0) were significantly increased in pSS relative to controls implying an association between both of these haplotypes and susceptibility to pSS.

TABLE 6
CR1 haplotype frequencies in pSS patients compared to controls.

| Haplotype | pSS | Controls | Odds Ratio (95% CI) |
|---|---|---|---|
| AH1 | 81 (35.2%) | 46 (23.5%) | 2.2 (1.4, 3.6)* |
| AH2 | 26 (11.3%) | 27 (13.8%) | 1.2 (0.7, 2.1) |
| AH3 | 37 (16.1%) | 19 (9.7%) | 2.6 (1.3, 5.0)* |
| Other | 86 (37.4%) | 104 (53.1%) | 1 |
| 2N | 230 | 196 | |

CR1 haplotype frequency distribution was significantly different between pSS patients and controls (χ² = 15.5, df = 3, p = 0.001), with relative increases observed in both AH1 and AH3 in pSS patients.

### Anti-Ro/La Autoantibody Subsets in pSS

Of 115 pSS patients, 18 (16%) were seronegative and 97 (84%) seropositive for anti-Ro/La autoantibodies. Seropositive Ro+La patients by ELISA were further subdivided into precipitating La, i.e. Ro+La (ppt+), or non-precipitating i.e. Ro+La (ppt−), on the basis of a precipitin line formed by anti-La antibodies on CIEP. Therefore, in addition to a seronegative subset, seropositive pSS patients were classified into one of three serological subsets: anti-Ro alone (18/115=16%), anti-Ro+La(ppt−) (19/115=17%), and anti-Ro+La(ppt+) (56/115=49%) which reflect differences in diversification of the autoantibody response (Rischmueller et al. 1998).

### CR1 Haplotypes in pSS Anti-Ro/La Subsets

CR1 haplotype frequencies differed significantly between the four serological subsets within pSS patients (χ²=21.4, df=9, p=0.011). Differences between seropositive and seronegative patients (χ²=8.2, df=3, p=0.042) and between the three seropositive subsets (χ²=12.1, df=6, p=0.059) both contributed substantially to this overall difference.

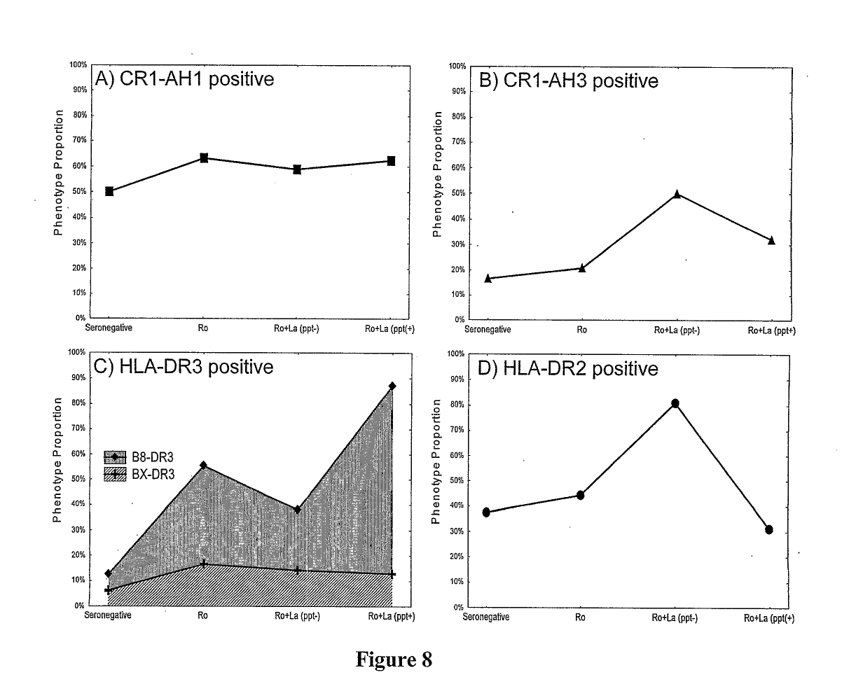

CR1 AH1 and AH3 phenotype frequencies by Ro/La subsets are depicted in FIG. 8. There is a modest, but consistent increase in the AHI phenotype frequency in all three seropositive subsets compared to the seronegative subset (˜60% vs 50%, FIG. 8A), in contrast to a phenotype frequency of 39% in the controls (data not shown). In contrast, the phenotype frequency of AH3 is relatively high in both Ro+La serological subsets, but most strikingly so in the Ro+La (ppt−) subset (FIG. 8B). The AH3 phenotype frequencies in the seronegative and anti-Ro subsets are comparable to that in the controls (17%, data not shown).

CR1 haplotypes and HLA

An association between both HLA-DR3 and HLA-DR2 and pSS is well established in Caucasians. We, and others (Gottenberg et al. 2003), have further dissected this association to demonstrate that the HLA class II associations are specific for seropositive pSS and further, HLA-DR3 and DR2 frequencies differ between autoantibody subsets reflecting differences in the diversification and regulation of the autoantibody response. This is analogous to the observed CR1 haplotype associations.

The phenotypic frequencies of HLA-DR3 and DR2 by Ro/La subsets are shown in FIG. 8. HLA DR3 is increased in all seropositive pSS subsets, most strikingly so in the anti-Ro+La (ppt+) subset (FIG. 8C). Moreover, this increase in DR3 is almost exclusively associated with the B8-DR3 haplotype. In contrast, DR2 is specifically associated with the anti-Ro+La (ppt−) serological subset (FIG. 8D). The high frequency of CR1 AH3 also observed in this subgroup (FIG. 8B) extends our previous observation that this is a distinct genetic subgroup within pSS. Ro+La (ppt−) autoantibodies are less polyclonal and of lower titre than Ro+La(ppt+) autoantibodies, and are associated with lower rheumatoid factor and serum IgG levels (Beer et al. 1996). Therefore, the different genetic associations between these two serological subsets are consistent with a quantitative, regulatory influence of both the MHC and CR1 regions on the autoantibody response.

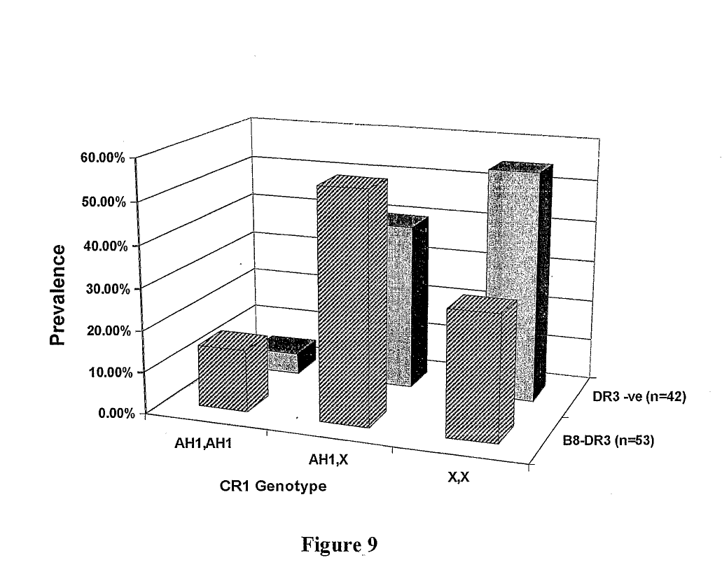

There was a significant positive association between AHI and the HLA B8-DR3 haplotype in pSS (χ=6.8, df=2, p=0.033, Table 7, FIG. 9). The AH1 association with B8-DR3 was significant for both AH1 homozygotes (OR 5.8, 95% CI 1.1,30.7) and AH1 heterozygotes (OR 2.5, 95% CI 1.1,5.9), and the magnitude of the odds ratios are consistent with a dosage effect i.e. the association with B8-DR3 was stronger with AH1 homozygotes than with AHI heterozygotes. There was no evidence of an association between AH1 and other DR3 haplotypes, nor with AH3 and any DR3 haplotypes. Therefore, the basis for the association between AH1 and B8-DR3 is most likely restricted to the 8.1 ancestral haplotype rather than other DR3 containing haplotypes. Interestingly, 8.1 contains only one, rather than 2 or more C4 genes and is therefore associated with relative C4 deficiency (Candore et al., 2002).

TABLE 7
CR1 AH1 genotype frequencies in HLA B8-DR3
positive and DR3 negative pSS patients.

| | HLA | | |
|---|---|---|---|
| AH1 Genotype | B8-DR3 | DR3 Neg | Odds Ratio (95% CI) |
| AH1, AH1 | 8 (15%) | 2 (5%) | 5.8 (1.1, 30.7)* |
| AH1, X | 29 (55%) | 17 (40%) | 2.5 (1.1, 5.9)* |
| XX | 16 (30%) | 23 (55%) | 1 |
| N | 53 | 42 | |

AH1 genotype frequency distribution was significantly different between B8-DR3 positive and DR3 negative pSS patients (χ² = 6.8, df = 2, p = 0.033). Both AH1 homozygotes and heterozygotes were over-represented in B8-DR3 positive patients in a dose dependent manner. “X” represents other, non-AH1, haplotypes.

The genes for C2 are also in the extended MHC region and type 1 C2 deficiency is encoded within the 18.1 haplotype which carries B18-DR2. However, only four B18-DR2 (from a total of 52 DR2) haplotypes were observed in this study. As expected, there was no evidence of an association between AH1 or AH3 and DR2 haplotypes.

### Discussion

The rationale of the GMT haplotyping approach is that sequence polymorphism is concentrated in regions which have been developed by local imperfect sequential duplication associated with indels and suppression of recombination. The method involves amplification of geometric elements which vary in size between duplicated segments and the subsequent profiles of PCR products of different sizes mark haplotypes of coding and non-coding sequences of hundreds of kilobases. GMT CR1 haplotyping has revealed extensive haplotypic polymorphism in this region (which also includes CR1-L, MCP and MCP-L genes) with more than 20 haplotypes defined, although the majority are rare.

In this Example we show that GMT CR1 haplotypes AH1 and AH3 are associated with pSS (Table 6), an autoimmune disease with a high prevalence of anti-nuclear Ro/La autoantibodies, and which shares both clinical and genetic susceptibility overlap with SLE. Similar to HLA haplotypes, CR1 haplotypes appear to exert a regulatory influence on the diversification and quantitation of the Ro/La autoantibody response in pSS patients (FIG. 8). Importantly, AHI was positively associated with HLA B8-DR3 in pSS patients (Table 7, FIG. 9). The basis for this association is most likely an epistatic effect between the CR1 receptor and C4, one of its ligands. The genes for C4 are in the extended MHC region. HLA B8-DR3 and a relative C4 insufficiency (C4A*Q0,C4B*1) (Candore et al. 2002) are both part of the 8.1 haplotype, which is strongly associated with a range of autoimmune diseases (Candore et al. 2002). The genetic structure of the C4 region is itself complex and highly polymorphic with both allelic and copy number variation of C4A and C4B genes (Blanchong et al. 2001).

We predict that both AH1 and AH3, associated with seropositive pSS, result in some form of CR1 and/or MCP dysfunction. There are genetically controlled differences in the level of CR1 expression, molecular weight (associated with differences in the number of C3b binding domains) and C4b binding affinity, which will all independently contribute to CR1 function. The CR1 haplotypic diversity and the potential for interaction with C4 allelic diversity compounds this complexity.

Ancestral haplotypes or “polymorphic frozen blocks” contain multiple genes, exhibit differences in their copy number and contain insertion/deletions in addition to coding region variation. Disease susceptibility could be a function of all of these differences which are captured by the GMT haplotyping approach and for which individual SNP analyses are uninformative.

In conclusion, the inventors have demonstrated that CR1 haplotypes are associated with the diversification/regulation of the Ro/La autoantibody response in pSS, an autoimmune disease with both clinical and genetic overlap with SLE. They have also demonstrated an interaction between HLA B8-DR3, a component of the autoimmune 8.1 haplotype and one of these CR1 haplotypes, the basis for which is most likely an epistatic effect between the CR1 receptor and its C4 ligand. In addition to systemic diseases associated with autoantibody production such as pSS and SLE, MHC 8.1 haplotype is also associated with a number of organ specific autoimmune diseases such as Type 1 diabetes, hypothyroidism, celiac disease, myasthenia gravis and multiple sclerosis.

### Example 6

### GMT markers for Complement Factor H (CFH) Haplotypes

The present inventors have developed GMT markers for Complement Factor H (CFH) haplotypes (1q32). The CFH gene is a member of the Regulator of Complement Activation (RCA) gene cluster and is located approx 11 Mb centromeric of CR1 and encodes a protein with twenty short concensus repeat (SCR) domains. This protein is secreted as a soluble factor and has an essential role in the regulation of complement activation, restricting this innate defense mechanism to microbial infections. Mutations in this gene have been associated with hemolytic-uremic syndrome (HUS) and chronic hypocomplementemic nephropathy. Alternate transcriptional splice variants, encoding different isoforms, have been characterized.

The following primers were developed for GMT analysis of CFH haplotypes.

(SEQ ID NO: 5)

| | |
|---|---|
| FHF1 | 5′ GCC TCT TGG TTT GAT TTT GG 3′ |

(SEQ ID NO: 6)

| | |
|---|---|
| FHR1 | 5′ CAG GGT CTA GCA TGA AGA GTA AAA 3′ |

(SEQ ID NO: 7)

| | |
|---|---|
| FHF4 | 5′ GCA AAC TCA ACA TTT CCC TAA CA 3′ |

(SEQ ID NO: 8)

| | |
|---|---|
| FHR4 | 5′ TGA TAC CAG GAG AAA TTG CAT 3′. |

The polymorphic elements are within intron 9 of the CFH gene and are separated by approximately 300 bp. The predicted amplicon products contained potential GMT elements as well as microsatellites.

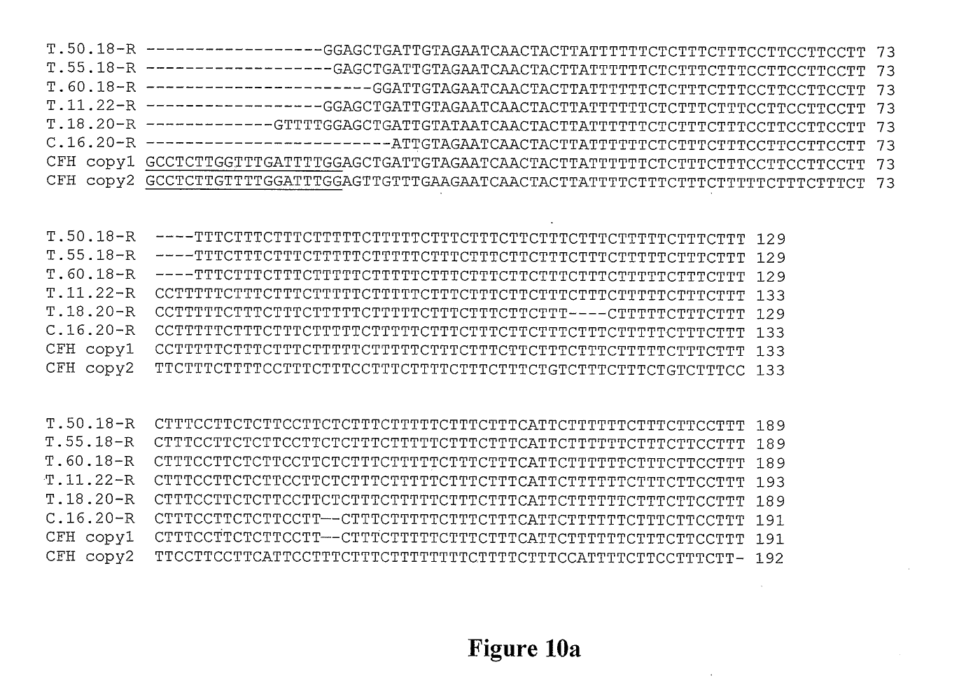

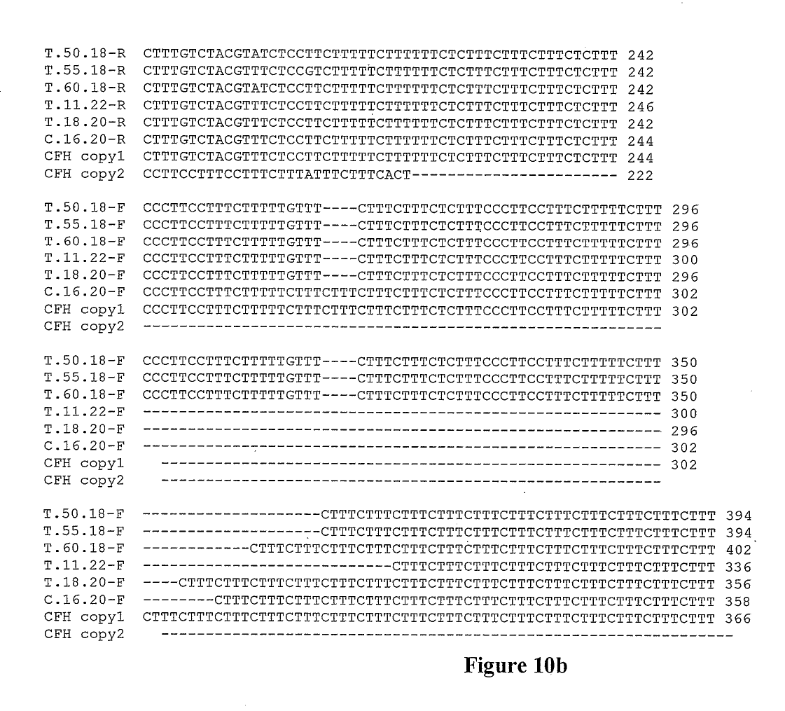

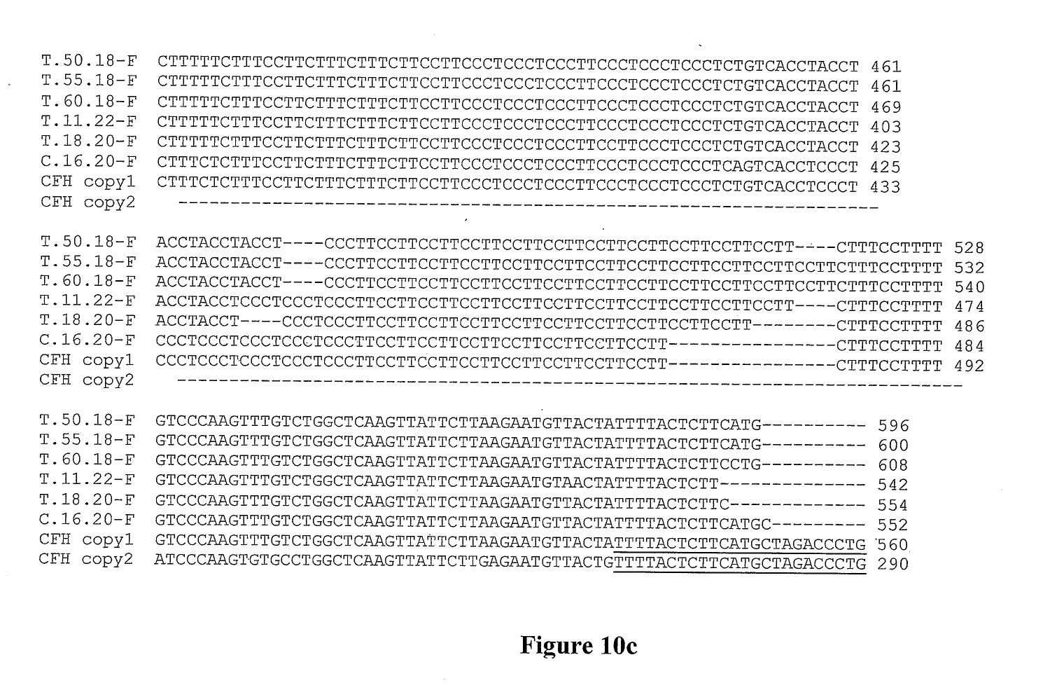

Each primer pair was expected to produce two products per haplotype, however, in each case one of the amplicons is highly conserved, and hence from each sample between 2 and 4 products were generated. Bands designated 11, 16, 18, 50, 55 and 60 were purified and sequenced. Alignment of the sequences showed that the major length polymorphism was primarily due to differences in two microsatellite (MS) units (CTTT and CCTT). Microsatellites are known to be less stable than GMT elements, and hence additional markers are now under evaluation. Nevertheless, in these examples there were additional indels within potential GMT elements (see FIG. 10) and the primers were tested in 5 three generation families to determine haplotypic segregation. In all but one case, mendelian segregation was demonstrated. In one individual, one of the FH4 alleles mutated from 23 to 22 (Family 1363, haplotype c) as would be expected for microsatellite mutation. Allowing for minor variations at each locus, 8 distinct haplotypes were identified in these 5 families.

The H402Y SNP was tested for all samples to further characterise the haplotypes. The segregation was consistent with the haplotypes defined assuming no recombination. Interestingly, this subdivided some of the haplotypes defined by the FH1 and FH4 primers. This showed the T SNP on all 9 haplotypes, but in addition, the 4 haplotypes with C had identical or similar FH1/4 alleles. Three out of the four C haplotypes had frequencies similar to the equivalent T haplotype, however, the C,(15-18), 1,2,(20-22) was the most common C haplotype and three times more frequent than the T equivalent. Within the families tested, the T and C haplotypes had frequencies of 0.66 and 0.34 respectively. These results suggest that the 402 SNP is unlikely to be a reliable marker of CFH haplotypes.

### Example 7

### Ancestral haplotypes of Complement Factor H: Comparison of Haplotyping and SNP Typing in Age-Related Macular Degeneration

### Materials and Methods

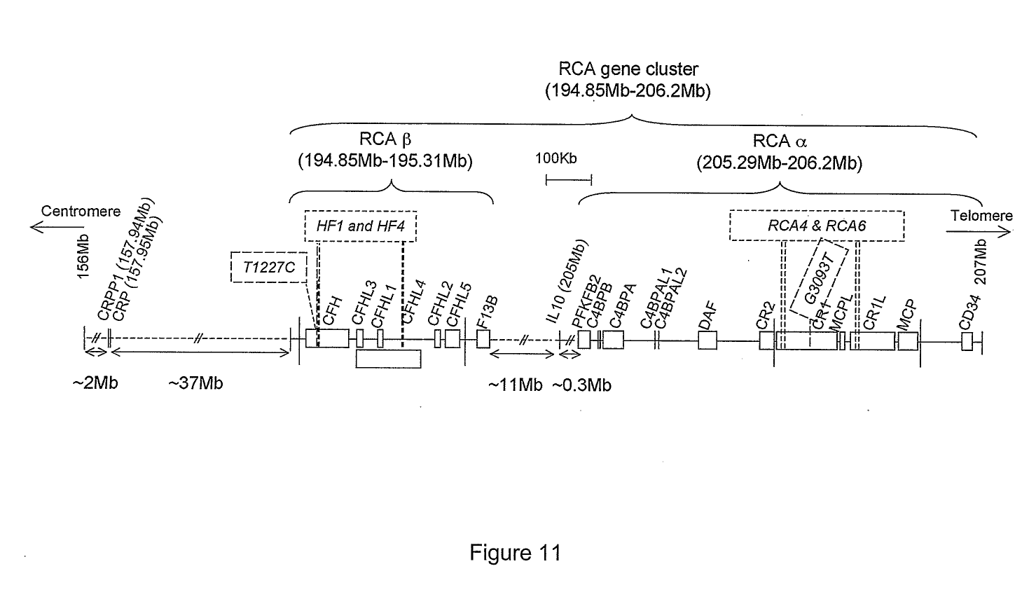

Within and around the RCA complex spanning some 13 megabases (Mb) of 1q there are genes such as CRP, IL-10 and complement receptors 1 and 2 with at least two large genomic blocks of approximately 500 kilobases (kb) at the telomeric (RCA alpha block) and centromeric (RCA beta block) ends (see FIG. 11). Both blocks contain duplicated genes important in binding, inactivating and clearing circulating immune complexes containing activated C3 and C4. The inactivation of these immune complexes controls further activation of the complement cascade and therefore the formation of the Membrane Attack Complex (MAC). CFH and its copies (CFHL1-5) are located within the RCA beta block.

The strategy of the GMT and the majority of the Materials and Methods have been described previously. Specific exceptions relating to the RCA beta block are described below.

The procedure used on this occasion involved the following steps:

1) Identification of Duplicons.

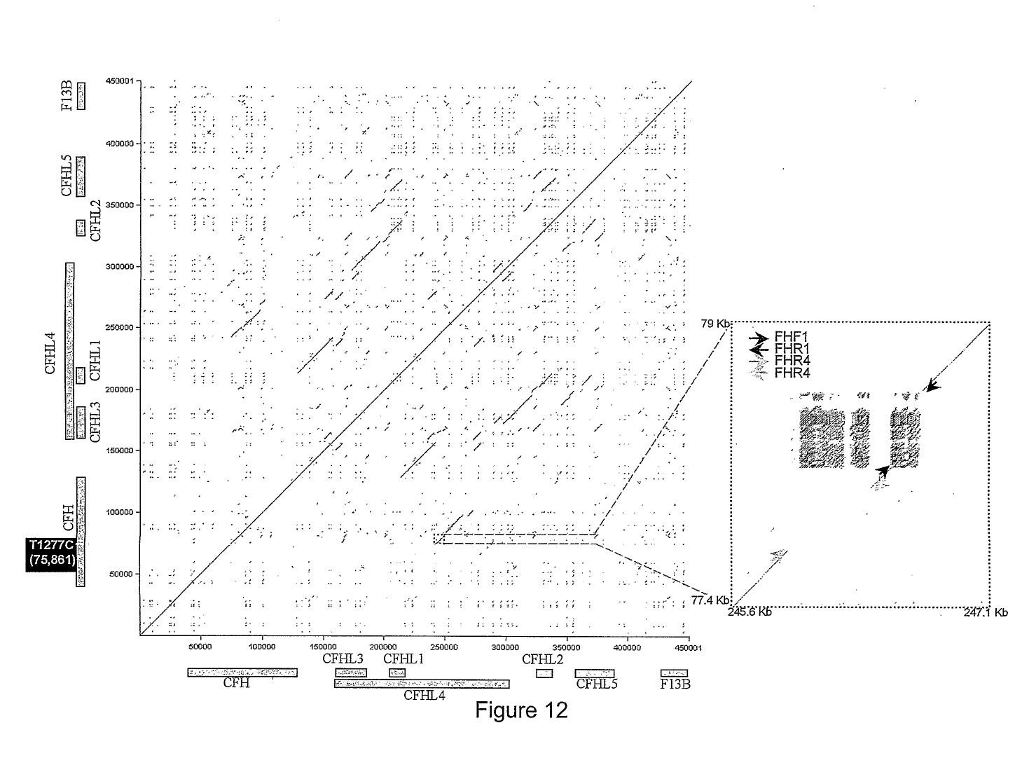

The genomic region designated RCA beta and containing CFH, CFHL1, CFHL2, CFHL3, CFHL4, CFHL5 and F13B at 1q32, was taken from the NCBI database (http://www.ncbi.nlm.nih.gov/) (position 47073731-47523731 on contig NT_{—}004487.18 (gi:88943682); accession numbers AL591604.6, AL049744.8, AL049741.8, BX248415.2, AL139418.9, AL353809.20). This sequence was compared against itself using Accelrys gene 2.0 (window size of 30 and hash value 6) to identify evidence of duplication (FIG. 12).

2) Selection of Primer Sites Present in all Duplicons.

FIG. 11 was examined for evidence of complex elements present in multiple duplicons. These regions were analysed in detail and screened for retroviral sequence using Repeatmasker (http://repeatmasker.org/cgi-bin/WEBRepeatMasker).

Duplicons at position 47,151,437-47,151,915 (CFH) and 47,319,604-47,320,203 (CFHL4), 47,151,937-47,152,496 (CFH) and 47,320,224-47,320,514 (CFHL4) of NT_{—}004487.18 were aligned using Accelrys gene 2.0. Primers were designed using Primer 3 (http://frodo.wi.mit.edu/cgi-bin/primer3/primer3).

Analysis of the in silico generated amplicons from the NCBI and Celera assemblies (http://www.ncbi.nlm.nih.gov/—NT_{—}004487.18 position 47073731-47523731 and NW_{—}926128.1 position 34954759-35404759 respectively) predicted that the duplicated elements are polymorphic when different individuals are compared.

RCA beta genotypes were defined by segregation analyses in five 3 generation families (Table 8). Three families (CEPH/Utah Pedigree 1362, CEPH/Amish Pedigree 884 and Venezuelan Pedigree 104) were obtained from Coriell Cell Repositories (http://ccr.coriell.org). Two local families (CYO1 and CYO2) have been previously described (McLure et al. 2005b). The 4AOH samples (http://www.ecacc.org.uk/) were obtained from in-house DNA stocks (Cattley et al. 2000). Forty seven living patients diagnosed with probable Alzheimer's disease, using NINCDS-ADRA criteria, were used (McKhann et al. 1984). Twenty samples from Aged-related Macular Degeneration patients were provided by The Lion's Eye Institute (Nedlands, Western Australia). These have been classified as AMD ‘wet’ or ‘dry’.

TABLE 8
CFH haplotypes of amplicon products from FH1 and FH4 primers
and T1277C SNP marker defined by segregation analysis.

| | | | Rela- | | | | |
|---|---|---|---|---|---|---|---|
| Lab No | ID | Family | tionship | FH1 | FH4 | AH | SNP |
| C04/00163D | | CYO2 | I 1a | 8 | 7 | 1 | C |
| C06/00372N | NA11994 | 1362 | MGF | 8 | 7 | | C |
| C06/00526D | NA13356 | 104 | PGM | 8 | 7 | | C |
| C04/00157M | | CYO1 | I 1 | 9 | 7 | | T |
| C06/00526D | NA13356 | 104 | PGM | 9 | 8 | | T |
| C04/00157M | | CYO1 | I 1 | 8 | 8 | | C |
| C04/00220C | | CYO1 | II 3a | 9 | 8 | | C |
| C0600/379J | NA05961 | 884 | PGM | 10 | 8 | | C |
| C06/00405Z | NA13055 | 104 | MGF | 4 | 7 | 3 | T |
| C06/00370A | NA11992 | 1362 | PGF | 4 | 7 | | C |
| C04/00163D | | CYO2 | I 1a | 5 | 7 | | C |
| C06/00407M | NA13057 | 104 | MGM | 5 | 7 | | T |
| C04/00156F | | CYO1 | I 1a | 5 | 8 | | T |
| C04/00162X | | CYO2 | I 1 | 4 | 4 | 5 | T |
| C04/00156F | | CYO1 | I 1a | 4 | 4 | | T |
| C06/00379J | NA05961 | 884 | PGM | 5 | 4 | | T |
| C06/00380S | NA05963 | 884 | PGF | 5 | 4 | | T |
| C04/00176Q | | CYO2 | II 1a | 4 | 5 | | T |
| C06/00373U | NA11995 | 1362 | MGM | 12 | 9 | 6 | T |
| C06/00392Y | NA06015 | 884 | MGM | 13 | 7 | | T |
| C04/00162X | | CYO2 | I 1 | 14 | 6 | | C |
| C06/00393E | NA11035 | 104 | PGF | 14 | 7 | | C |
| C06/00407M | NA13057 | 104 | MGM | 14 | 9 | | C |
| C04/00176Q | | CYO2 | II 1a | 7 | 8 | 2 | T |
| C06/00391R | NA06013 | 884 | MGF | 7 | 8 | | T |
| C06/00392Y | NA06015 | 884 | MGM | 7 | 8 | | T |
| C06/00393E | NA11035 | 104 | PGF | 7 | 9 | | T |
| C06/00373U | NA11995 | 1362 | MGM | 8 | 3 | 7 | T |
| C06/00372N | NA11994 | 1362 | MGF | 10 | 3 | | T |
| C06/00391R | NA06013 | 884 | MGF | 10 | 3 | | C |
| C04/00220C | | CYO1 | II 3a | 9 | 3 | | C |
| C06/00405Z | NA13055 | 104 | MGF | 17 | 7 | 4 | T |
| C06/00371G | NA11993 | 1362 | PGM | 19 | 7 | | T |
| C06/00380S | NA05963 | 884 | PGF | 7 | 5 | 11 | T |
| C06/00370A | NA11992 | 1362 | PGF | 7 | 5 | | T |
| C06/00371G | NA11993 | 1362 | PGM | 3 | 9 | 9 | T |

The alleles for each primer pair have been numbered sequentially according to size.

3) Assignment of Haplotypes.

FH1 and FH4 amplicon products were assigned numbers based on the respective size (as described in McLure et al. (2005b)). In the CEPH families, the haplotypes of the paternal grandfather, paternal grandmother, maternal grandfather and maternal grandmother within each family were assigned ab, cd, ef and gh respectively. In the case of the CYO families, the ef haplotypes were assigned to the spouse in the second generation. These haplotypes were then used to manually genotype other individuals. In situations where different haplotypes from the reference families could be assigned with alternative combinations, the haplotype with the highest frequency was used.

### Amplification and Analysis of CFH and CFHL4 (HGEs)

The following primers were used.

FH1

(SEQ ID NO: 5)

| | |
|---|---|
| FHF1 | 5′ GCC TCT TGG TTT GAT TTT GG 3′ |
| and | |

(SEQ ID NO: 6)

| | |
|---|---|
| FHR1 | 5′CAG GGT CTA GCA TGA AGA GTA AAA 3′. |
| FH4 | |

(SEQ ID NO: 7)

| | |
|---|---|
| FHF4 | 5′ GCA AAC TCA ACA TTT CCC TAA CA 3′ |
| and | |

(SEQ ID NO: 8)

| | |
|---|---|
| FHR4 | 5′ TGA TAC CAG GAG AAA TTG CAT 3′. |

PCR reactions were performed in a 96-well Palm Cycler (Corbett Research) in 20 μlvolumes using 100 ng of template DNA, 1.3 U Taq Polymerase (Fisher Biotec), 10 pmol of the forward and reverse FH primers, 200 μM of each dNTP, 2 mM MgCl₂ and 1×PCR buffer (Fisher Biotec). For the FH1 primers the samples were denatured at 94° C. for 5 min, followed by 30 cycles each comprising 30 seconds at 94° C., 45 seconds at 60° C. and 45 seconds at 72° C. The last cycle was followed by an additional extension for 5 minutes at 72° C. The conditions were the same for the FH4 primers with the exception that the annealing temperature was 58° C.

The separation and detection of the haplotype products was done with the Corbett Research GS-3000 automated gel analysis system. One microlitre of PCR product was mixed with 1 μl of loading buffer containing Puc19 molecular weight ladder. One microlitre of the PCR sample and loading buffer mixture was then added to a 32 cm long, 48 well, 4% polyacrylamide, ultra-thin gel and pulsed for 10 seconds at 2400 V. Excess sample was then flushed and the gel was run at 2000 V for 180 minutes.

The gel image was analysed using Bio-Rad Quantity One 1-D gel analysis software. Lanes were defined, amplicons detected and standards assigned. Densimetric profiles were generated and lanes were aligned using the internal Mid B 200 bp ladder (Fisher Biotec, Perth Western Australia).

### Band Purification and Sequencing

PCR products were analysed using a 2% agarose gel. Six Individual FH1 bands (7, 9, 10, 18, 19 and 20) were cut from the gel and purified using GFX PCR Gel Band Purification Kit (Amersham Biosciences). The purified products were amplified as above and sequenced.

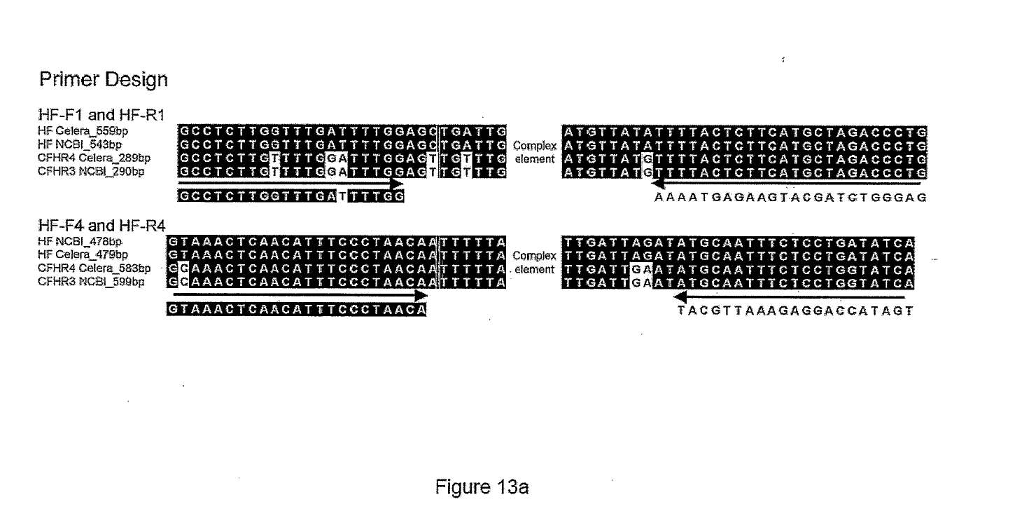

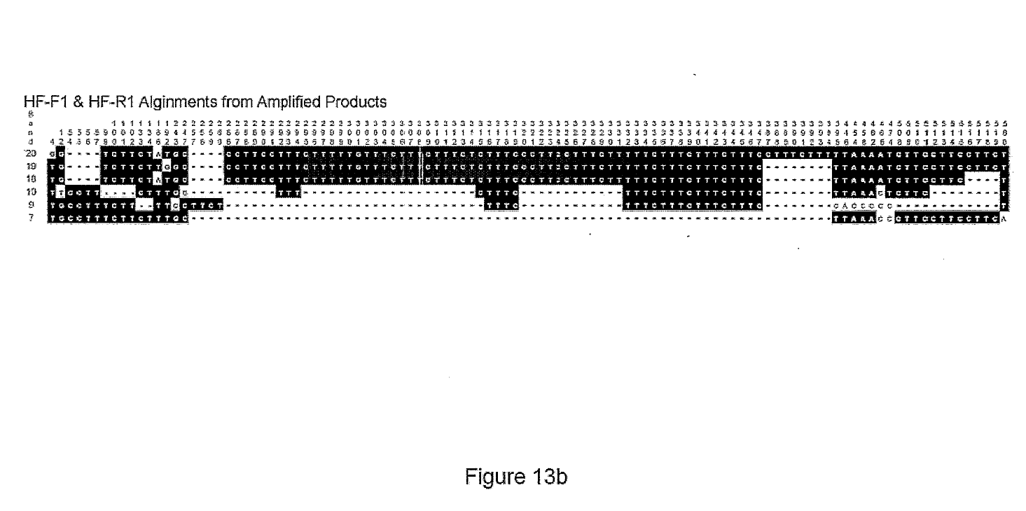

Sequencing reactions were performed using the FH1 primers listed above. Alignments of sequenced amplicons are shown in FIG. 13b.

### T1277C and Y402H SNP Detection

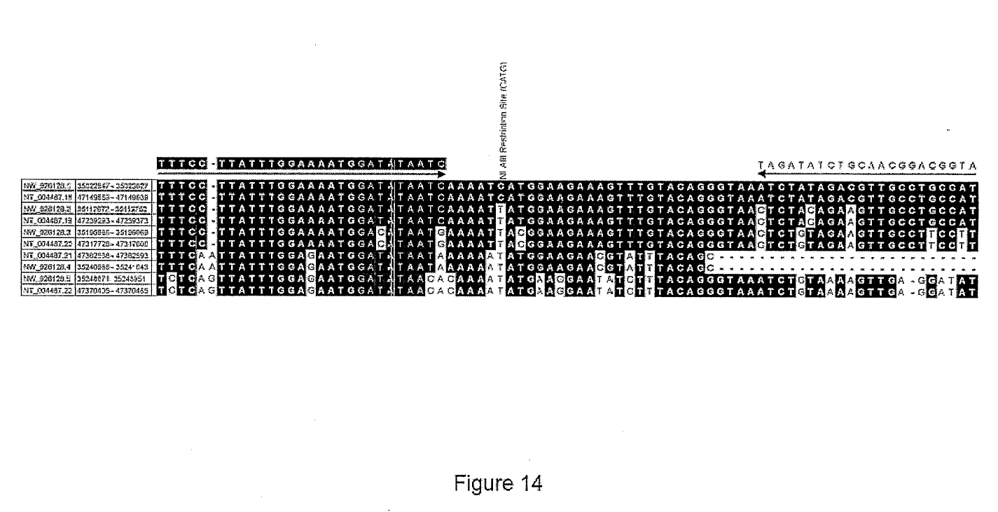

The sequence for CFH Exon 9 was selected and analysed against the genome to identify homologous copies. Homologous sequences from four FHR genes were identified. The five NCBI (http://www.ncbi.nlm.nih.gov/; contig NT_{—}004487.18, positions: 47,149,559-47,149,639; 47,239,293-47,239,373; 47,317,728-47,317,808; 47,362,538-47,362,593; 47,370,405-47,370,485) and five Celera (http://www.ncbi.nlm.nih.gov/; contig NW_{—}926128.1 positions: 35,022,947-35,023,027; 35,112,672-35,112,752; 35,195,989-35,196,069; 35,240,988-35,241,043; 35,248,871-35,248,951) sequences were aligned and sequence specific primers designed to bind and amplify only CFH exon 9 (FIG. 14). PCR conditions were as above, except the primer Tm was 60.5° C.

Digestion was performed using NLA III (New England Biolabs), which cuts at 1277C but not 1277T. Digestion mix was performed as recommended by the manufacturer. Digested products were separated using the Corbett Research GS-3000, using the same conditions as described in McLure et al. (2005b).

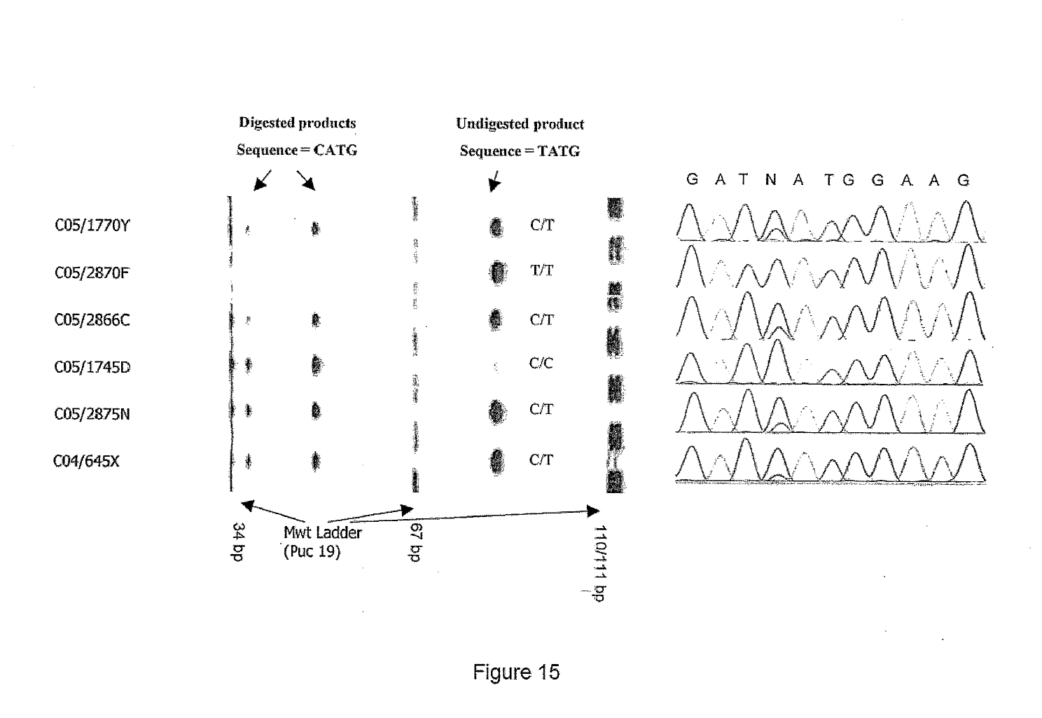

Homozygotes 1277T individuals were identified by a single band 8 lbp in length whereas homozygote 1277C had 2 bands, one 37 bp in length and the other 44 bp (FIG. 15). Heterozygotes contained all three bands. Homozygotes and heterozygote assignments were confirmed by sequencing CFH exon 9 on 6 samples (FIG. 15).

### Results

### Frequency of T1277C

Twenty seven of the 94 control haplotypes carry the C allele (29%) compared with 17/40 (43%) of the AMD group (p=0.09) and 10/20 (50%) of the WET subgroup (p=0.06).

### Frequency of RCA Beta Haplotypes

The products from the FH1 and FH4 primers are highly polymorphic with 20 and 11 products observed respectively.

Haplotyping of the 18 members of 5 three generation families is shown in Table 8. Due to the limited numbers at this time and to be conservative, products which are similar in size were not distinguished resulting in the designation of only 9 combinations which occurred as putative ancestral haplotypes RCA beta 1 to 9. AH 1 has a frequency of 22%.

Unrelated control samples were tested with the FH primers so that hap lotypes could be assigned as described in the Materials and Methods. In all 29 individuals, at least one of the nine putative AHs is present. A further three putative AHs (RCA beta 10, 11, 12) were assigned because of their relatively high frequency. The most frequent haplotype, (AH1), is present in 26% of the combined control group (n=94).

An additional control group of forty seven individuals with Alzheimer's disease but not AMD was tested with the FH primers. All haplotypes could be assigned assuming the same 12 putative AHs. Further, the frequency of AH1 is 26% (18/70).

The 12 AHs were then assigned in patients with AMD. The frequency of AH 1 is 60% (p=0.004) and 40% (p=0.15) in the wet and dry subgroups respectively which compares to 22-26% in the various control groups. Interestingly, all of the 10 patients with the wet form have at least one copy of AH1 in contrast to only six of the 10 patients with the dry form and 6 of the 18 family controls (Table 9).

### Comparison of T1277C and RCA Beta Haplotypes

Overall, the C allele is present in 29% of the control haplotypes.

Each example of a particular ancestral haplotype is expected to carry the same sequence. Indeed, all examples of RCA beta haplotypes 4, 5, 10, 11 and 12 (n=24) carry a T at 1277. Surprisingly however, AHs 1, 2, 3, 6, 7, 8, and 9 carry a C in some examples but a T in others. The 1277C allele is present in 26/53 (49%) of AHs 1, 3, 6, 7, 8 and 9 compared to 1/18 (0.06%) of AH2. This diversity suggests that at least AHs 1, 2, 3, 6, 7, 8 and 9 will be split into two or more variants as further subjects and markers are studied and that each new haplotype will carry either C or T. Alternatively, the 1277 site could be mutating more rapidly than the background sequence although this seems unlikely (see FIG. 14). In either case, the AH is more relevant than the SNP.

TABLE 9
Ancestral haplotypes of CFH using GMT and association
with progression from dry to wet AMD.

| | | AMD | CFH-AH | | | | SNP | |
|---|---|---|---|---|---|---|---|---|
| Lab No | ID | presentation | 1 | 2 | 3 | other | T | C |
| C05/2876U | AMD - | wet | 2 | | | | 2 | |
| | P5117 | | | | | | | |
| C05/2875N | AMD - | wet | 2 | | | | 1 | 1 |
| | P3844 | | | | | | | |
| C05/2872T | AMD - | wet | 1 | | 1 | | | 2 |
| | M7050 | | | | | | | |
| C05/2874G | AMD - | wet | 1 | | 1 | | | 2 |
| | P3753 | | | | | | | |
| C05/2878H | AMD - | wet | 1 | 1 | | | 1 | 1 |
| | V1393 | | | | | | | |
| C05/2877B | AMD - | wet | 1 | | 1 | | 1 | 1 |
| | P4815 | | | | | | | |
| C05/2869X | AMD - | wet | 1 | | | 1 | 1 | 1 |
| | O1278 | | | | | | | |
| C05/2873A | AMD - | wet | 1 | | | 1 | | 2 |
| | P3856 | | | | | | | |
| C05/2870F | AMD - | wet | 1 | | | 1 | 2 | |
| | M537 | | | | | | | |
| C05/2871M | AMD - | wet | 1 | 1 | | | 2 | |
| | N1597 | | | | | | | |
| C05/2859E | AMD - | dry | 2 | | | | | 2 |
| | B8465 | | | | | | | |
| C05/2866C | AMD - | dry | 2 | | | | 1 | 1 |
| | C7630 | | | | | | | |
| C05/2864P | AMD - | dry | 1 | 1 | | | | 2 |
| | K1822 | | | | | | | |
| C05/2867J | AMD - | dry | 1 | | | 1 | | 2 |
| | H6226 | | | | | | | |
| C05/2860N | AMD - | dry | 1 | | | 1 | 2 | |
| | P5136 | | | | | | | |
| C05/2861U | AMD - | dry | 1 | | | 1 | 2 | |
| | N1915 | | | | | | | |
| C05/2863H | AMD - | dry | | 1 | | 1 | 2 | |
| | M3949 | | | | | | | |
| C05/2868Q | AMD - | dry | | 1 | | 1 | 2 | |
| | H6901 | | | | | | | |
| C05/2865W | AMD - | dry | | 1 | | 1 | 2 | |
| | K3239 | | | | | | | |
| C05/2862B | AMD - | dry | | 1 | | 1 | 2 | |
| | O1544 | | | | | | | |

### Discussion

Contrary to previous understanding, we have shown that there is extensive polymorphism in, and around, CFH. Based on experience with CR1 and the MHC, the greater yield of polymorphism is likely to be due to the use of the GMT approach (see FIG. 11) which has proved to be superior to combining SNPs.

The recognition of the same 13 AHs in the various groups provides strong evidence for their relatively high population frequency and therefore their remote ancestry and faithful inheritance over many generations. Each AH is a marker for many kilobases of polymorphic sequence no doubt including many genes and innumerable SNPs. It follows that haplotyping will be a useful method of examining associations between RCA polymorphisms and inflammatory diseases such as AMD. Thus, haplotyping can be compared to SNP typing.

Using a combination of sequencing and amplicon digestion, the T1277C results were clear cut and indicate that the digestion method is robust and useful as a single approach. The frequencies of T1277C are consistent with previous reports in Caucasoid populations and patients (Hageman et al. 2005; Donoso et al. 2006; Grassi et al. 2006) and again confirm that there are genetic factors influencing susceptibility to AMD and possibly progression to the wet form. Note, however, that the predictive values are too low to be of immediate clinical value.

The results of haplotyping are similar in some respects but interesting from several perspectives. Firstly, if confirmed in larger studies, haplotyping has the promise of increasing predictive values. As illustrated by the present data, a negative result for AH1 may indicate that progression to the wet form is unlikely.

Secondly, T1277C and haplotyping provide different information. Although most examples of AH1 carry the C allele, this is not always the case. Indeed it is possible that the T1277C results are secondary to the AH1 association. Some support for this interpretation is provided by previous demonstration that more than one SNP may be relevant (Haines et al. 2005; Klein et al. 2005; Edwards et al. 2005; Hageman et al. 2005, Despriet et al. 2006; Okamoto et al. 2006). The splits of AH1 which carry the C allele may be particularly powerful and may provide a means of distinguishing between C alleles which are either important or irrelevant. In this way it will be possible to increase predictive values.

Thirdly, the association with AH1, irrespective of T12277C, strongly suggests that there are influences which could be within, or remote to, CFH. In other words, the haplotypes may mark very extensive sequences which may extend well beyond CFH and may reflect alleles of adjacent genes.

Irrespective of the explanation for the association, the present findings show that progression from wet to dry may be predicted by genetic testing. For example, AH1 appears, in this sample, to be a sine qua non for progression.

It will be appreciated by persons skilled in the art that numerous variations and/or modifications May be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Beer et al. (1996) Clin Immunol Immunopathol 79: 314-318.
Birmingham et al. (2003) Immunology 108:531-538.
Blanchong et al. (2001) Int Immunopharmacol 1:365-392.
Candore et al. (2002) Autoimmun Rev 1:29-35.
Cattley et al. (2000) European Journal of Immunogenetics 27: 397-426.
Dawkins et al. (1999) Immunological Reviews 167:275-304.
de Cordoba et al. (1999) Molecular Immunology 36:803-808.
Despriet et al. (2006) Jama 296:301-309.
Donoso et al. (2006) Surv Opthalmol 51:137-152.
Edwards et al. (2005) Science 308:421-424.
Grassi et al. (2006) Hum Mutat Epub.
Gottenberg et al. (2003) Arthritis Rheum 48: 2240-2245.
Hageman et al. (2005) Proc Natl Acad Sci USA 102:7227-7232.
Haines et al. (2005) Science 308:419-421.
Heine-Suner et al. (1997) Immunogenetics, 45:422-427.
Hourcade et al. (1989) Ad Immunol 45:381-416.
Klein et al. (2005) Science 308:385-389.
Longman-Jacobsen et al. (2003) Gene 312:257-261.
McKhann et al. (1984) Neurology 34:939-944.
McLure et al. (2004a) Journal of Molecular Evolution 59:143-157.
McLure et al. (2004b) Immunogenetics 56:631-638.
McLure et al. (2005a) Human Immunology 66:258-273.
McLure et al. (2005b) Immunogenetics 57:805-815.
Moulds et al. (2001) Blood 97:2879-2885.
Needleman and Wunsch (1970) Journal of Molecular Biology, 48:443-453.
Okamoto et al. (2006) Mol Vis 12:156-158.
Rischmueller et al. (1998) Clin. Exp. Immunol. 111:365-371.
Sonnhammer and Durbin (1995) Gene 167:GC1-10.
Vitali et al. (2002) Ann Rheum Dis, 2002. 61:554-558.
Xiang et al. (1999) Journal of Immunology 163:4939-4945.

1.-41. (canceled)

42. A method of identifying a polymorphism linked and/or responsible for, at least in part, an individuals susceptibility or predisposition to age-related macular degeneration, the method comprising
i) analysing the genotype at one or more loci of the RCA gene cluster on 1q32 of the human genome of individuals with age-related macular degeneration,
ii) analysing the genotype at one or more loci of the RCA gene cluster on 1q32 of the human genome of individuals who do not have age-related macular degeneration, and
iii) identifying a polymorphism linked and/or responsible for, at least in part, an individuals susceptibility to age-related macular degeneration,
wherein the polymorphism is not a polymorphism of the complement factor H gene.

43. A method of determining whether an individual is susceptible or predisposed to age-related macular degeneration, the method comprising analysing the genotype of the individual within a multigene cluster comprising genes encoding complement control proteins, and wherein the method comprises screening the individual for a haplospecific geometric element (HGE) linked to age-related macular degeneration and wherein said HGE comprise halospecific sequences which are specific for a particular ancestral haplotype, and wherein the sequences flanking said HGE are substantially conserved between ancestral haplotypes.

44. A method of diagnosing whether an individual has age-related macular degeneration, the method comprising analysing the genotype of the individual within a multigene cluster comprising genes encoding complement control proteins, and wherein the method comprises screening the individual for a. haplospecific geometric element linked to age-related macular degeneration.

45. The method of claim 43, wherein the multigene cluster is located on 1q32 of the human genome.

46. The method of claim 43, wherein the method comprises screening the individual for a polymorphism identified using a method according to claim 42.

47. The method of claim 43, wherein the haplospecific 15 geometric elements are present in the complement factor H and the complement factor HL4 genes.

48. The method of claim 43, wherein the method comprises
i) amplifying a region of the complement factor H and the complement factor HL4 genes using at least one set of oligonucleotide primers comprising the following sequences
(SEQ ID NO: 5)

| | |
|---|---|
| a) | 5′ GCC TCT TGG TTT GAT TTT GG 3′ |

(SEQ ID NO: 6)

| | |
|---|---|
| and | 5′ CAG GGT CTA GCA TGA AGA GTA AAA 3′, |

(SEQ ID NO: 7)

| | |
|---|---|
| b) | 5′ GCA AAC TCA ACA TTT CCC TAA CA 3′ |

(SEQ ID NO: 8)

| | |
|---|---|
| and | 5′ TGA TAC CAG GAG AAA TTG CAT 3′, |
| and | |

ii) analysing the amplification products to determine the ancestral haplotype of the individual.

49. The method of claim 48, wherein step ii) comprises analysing the size of the amplification products.

50. A method of determining whether an individual is susceptible or predisposed to progress from dry age-related macular degeneration to wet age-related macular degeneration, the method comprising analysing the genotype of the individual within a multigene cluster comprising genes encoding complement control proteins, and wherein the method comprises screening the individual for a haplospecific geometric element (HGE) linked to age-related macular degeneration, and wherein said HGE comprise haplospecific sequences which are specific for a particular ancestral haplotype, and wherein the sequences flanking said HGE are substantially conserved between ancestral haplotypes.

51. The method of claim 50, wherein the haplospecific geometric elements are present in the complement factor H and the complement factor HL4 genes.

52. The method of claim 51, wherein the method comprises
i) amplifying a region of the complement factor H and the complement factor HL4 genes using at least one set of oligonucleotide primers comprising the following sequences
(SEQ ID NO:5)

| | |
|---|---|
| a) | 5′ GCC TCT TGG TTT GAT TTT GG 3′ |

(SEQ ID NO: 6)

| | |
|---|---|
| and | 5′ CAG GGT CTA GCA TGA AGA GTA AAA 3′, |

(SEQ ID NO: 7)

| | |
|---|---|
| b) | 5′ GCA AAC TCA ACA TTT CCC TAA CA 3′ |

(SEQ ID NO: 8)

| | |
|---|---|
| and | 5′ TGA TAC CAG GAG AAA TTG CAT 3′, |
| and | |

ii) analysing the amplification products to determine the ancestral haplotype of the individual.

53. The method of claim 52, wherein step ii) comprises analysing the size of the amplification products.

54. The method of claim 53, wherein the presence of ancestral haplotype 1 (AHI) indicates that the individual has a greater chance of progressing from dry age-related macular degeneration to wet age-related macular degeneration than an individual lacking AHI.

55. An oligonucleotide primer for use in performing a genomic matching technique for diagnosing whether an individual has, is susceptible to or predisposed to age-related macular degeneration, wherein the primer can be used to amplify a region of a multigene cluster comprising genes encoding complement control proteins.

56. The oligonucleotide primer of claim 55, wherein the primer is selected from:
a) an oligonucleotide comprising a sequence selected from: 5′ GCC TCT TGG TTT GAT TTT GG 3′ (SEQ ID NO:5), 5′ CAG GGT CTA GCA TGA AGA GTA AAA 3′ (SEQ ID NO:6), 5′ GCA MC TCA ACA TTT CCC TM CA 3′ (SEQ ID NO:7) and 5′ TGA TAC CAG GAG AAA TTG CAT 3′ (SEQ ID NO:8),
b) an oligonucleotide comprising a sequence which is the reverse complement of any oligonucleotide provided in a), and
c) a variant of a) or b) which can be used to amplify the same region of the human genome as any one of the oligonucleotides of a) or b).

57. A composition comprising an oligonucleotide of claim 55 and an acceptable carrier.

58. A kit comprising an oligonucleotide of claim 55.

59. The method of claim 43, wherein the method comprises performing the genomic matching technique.


SEQUENCE LISTING (18 sequences)

SEQ ID NO: 1 - DNA, 22 bp, Artificial
  Oligonucleotide primer
aattccaaattggcctggttga

SEQ ID NO: 2 - DNA, 23 bp, Artificial
  Oligonucleotide primer
ccttccctttgagatgtggaaca

SEQ ID NO: 3 - DNA, 26 bp, Artificial
  Oligonucleotide primer
gtcagcttggattgcccttggttcta

SEQ ID NO: 4 - DNA, 25 bp, Artificial
  Oligonucleotide primer
cctgggcaacaaagcaagacattgt

SEQ ID NO: 5 - DNA, 20 bp, Artificial
  Oligonucleotide primer
gcctcttggtttgattttgg

SEQ ID NO: 6 - DNA, 24 bp, Artificial
  Oligonucleotide primer
cagggtctagcatgaagagtaaaa

SEQ ID NO: 7 - DNA, 23 bp, Artificial
  Oligonucleotide primer
gcaaactcaacatttccctaaca

SEQ ID NO: 8 - DNA, 21 bp, Artificial
  Oligonucleotide primer
tgataccaggagaaattgcat

SEQ ID NO: 9 - DNA, 20 bp, Artificial
  Oligonucleotide primer
aattccaaattggcctggtt

SEQ ID NO: 10 - DNA, 26 bp, Artificial
  Oligonucleotide primer
aaactttaactttgagatgtggaaca

SEQ ID NO: 11 - DNA, 568 bp, Homo sapiens
ggagctgattgtagaatcaactacttattttttctctttctttccttccttcctttttctttctttctttttctttttctttctttcttctttctttctttttctttctttctttccttctcttccttctctttctttttctttctttcattcttttttctttcttcctttctttgtctacgtatctccttctttttcttttttctctttctttctttctctttcccttcctttctttttgtttctttctttctctttcccttcctttctttttctttcccttcctttctttttgtttctttctttctctttcccttcctttctttttctttctttctttctttctttctttctttctttctttctttctttctttctttttctttccttctttctttcttccttccctccctcccttccctccctccctctgtcacctacctacctacctacctcccttccttccttccttccttccttccttccttccttccttccttctttccttttgtcccaagtttgtctggctcaagttattcttaagaatgttactattttactcttcatg

SEQ ID NO: 12 - DNA, 571 bp, Homo sapiens
gagctgattgtagaatcaactacttattttttctctttctttccttccttcctttttctttctttctttttctttttctttctttcttctttctttctttttctttctttctttccttctcttccttctctttctttttctttctttcattcttttttctttcttcctttctttgtctacgtttctccgtctttttcttttttctctttctttctttctctttcccttcctttctttttgtttctttctttctctttcccttcctttctttttctttcccttcctttctttttgtttctttctttctctttcccttcctttctttttctttctttctttctttctttctttctttctttctttctttctttctttctttttctttccttctttctttcttccttccctccctcccttccctccctccctctgtcacctacctacctacctacctcccttccttccttccttccttccttccttccttccttccttccttccttctttccttttgtcccaagtttgtctggctcaagttattcttaagaatgttactattttactcttcatg

SEQ ID NO: 13 - DNA, 575 bp, Homo sapiens
ggattgtagaatcaactacttattttttctctttctttccttccttcctttttctttctttctttttctttttctttctttcttctttctttctttttctttctttctttccttctcttccttctctttctttttctttctttcattcttttttctttcttcctttctttgtctacgtatctccttctttttcttttttctctttctttctttctctttcccttcctttctttttgtttctttctttctctttcccttcctttctttttctttcccttcctttctttttgtttctttctttctctttcccttcctttctttttctttctttctttctttctttctttctttctttctttctttctttctttctttctttctttttctttccttctttctttcttccttccctccctcccttccctccctccctctgtcacctacctacctacctacctcccttccttccttccttccttccttccttccttccttccttccttccttctttccttttgtcccaagtttgtctggctcaagttattcttaagaatgttactattttactcttcctg

SEQ ID NO: 14 - DNA, 510 bp, Homo sapiens
ggagctgattgtagaatcaactacttattttttctctttctttccttccttccttcctttttctttctttctttttctttttctttctttcttctttctttctttttctttctttctttccttctcttccttctctttctttttctttctttcattcttttttctttcttcctttctttgtctacgtttctccttctttttcttttttctctttctttctttctctttcccttcctttctttttgtttctttctttctctttcccttcctttctttttctttctttctttctttctttctttctttctttctttctttctttttctttccttctttctttcttccttccctccctcccttccctccctccctctgtcacctacctacctacctccctccctcccttccttccttccttccttccttccttccttccttccttccttctttccttttgtcccaagtttgtctggctcaagttattcttaagaatgtaactattttactctt

SEQ ID NO: 15 - DNA, 528 bp, Homo sapiens
gttttggagctgattgtataatcaactacttattttttctctttctttccttccttccttcctttttctttctttctttttctttttctttctttcttctttctttttctttctttctttccttctcttccttctctttctttttctttctttcattcttttttctttcttcctttctttgtctacgtttctccttctttttcttttttctctttctttctttctctttcccttcctttctttttgtttctttctttctctttcccttcctttctttttctttctttctttctttctttctttctttctttctttctttctttctttctttctttctttctttctttttctttccttctttctttcttccttccctccctcccttccttccctccctctgtcacctacctacctacctccctcccttccttccttccttccttccttccttccttccttccttctttccttttgtcccaagtttgtctggctcaagttattcttaagaatgttactattttactcttc

SEQ ID NO: 16 - DNA, 518 bp, Homo sapiens
attgtagaatcaactacttattttttctctttctttccttccttccttcctttttctttctttctttttctttttctttctttcttctttctttctttttctttctttctttccttctcttccttctttctttttctttctttcattcttttttctttcttcctttctttgtctacgtttctccttctttttcttttttctctttctttctttctctttcccttcctttctttttctttctttctttctttctctttcccttcctttctttttctttctttctttctttctttctttctttctttctttctttctttctttctttctttctttctttctctttccttctttctttcttccttccctccctcccttccctccctccctcagtcacctccctccctccctccctccctcccttccttccttccttccttccttccttccttctttccttttgtcccaagtttgtctggctcaagttattcttaagaatgttactattttactcttcatgc

SEQ ID NO: 17 - DNA, 560 bp, Homo sapiens
gcctcttggtttgattttggagctgattgtagaatcaactacttattttttctctttctttccttccttccttcctttttctttctttctttttctttttctttctttcttctttctttctttttctttctttctttccttctcttccttctttctttttctttctttcattcttttttctttcttcctttctttgtctacgtttctccttctttttcttttttctctttctttctttctctttcccttcctttctttttctttctttctttctttctctttcccttcctttctttttctttctttctttctttctttctttctttctttctttctttctttctttctttctttctttctttctttctttctctttccttctttctttcttccttccctccctcccttccctccctccctctgtcacctccctccctccctccctccctcccttccttccttccttccttccttccttccttctttccttttgtcccaagtttgtctggctcaagttattcttaagaatgttactattttactcttcatgctagaccctg

SEQ ID NO: 18 - DNA, 290 bp, Homo sapiens
gcctcttgttttggatttggagttgtttgaagaatcaactacttattttctttctttctttttctttctttctttctttcttttcctttctttcctttcttttctttctttctgtctttctttctgtctttccttccttccttcattcctttctttcttttttttcttttctttccattttcttcctttcttccttcctttcctttctttatttctttcactatcccaagtgtgcctggctcaagttattcttgagaatgttactgttttactcttcatgctagaccctg